SEQUENCE LISTING (13 sequences)

SEQ ID NO: 1 - protein, 5 aa, synthetic construct
SYVMH

SEQ ID NO: 2 - protein, 6 aa, synthetic construct
NPYNDG

SEQ ID NO: 3 - protein, 12 aa, synthetic construct
GTYYYGTRVFDY

SEQ ID NO: 4 - protein, 16 aa, synthetic construct
RSSKSLQNVNGNTYLY

SEQ ID NO: 5 - protein, 7 aa, synthetic construct
RMSNLNS

SEQ ID NO: 6 - protein, 9 aa, synthetic construct
MQHLEYPIT

SEQ ID NO: 7 - protein, 121 aa, synthetic construct
EVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSS

SEQ ID NO: 8 - protein, 112 aa, synthetic construct
DIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIK

SEQ ID NO: 9 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVQFNWYVDGVEVHNAKTKPREEQFNSTFRVVSVLTVVHQDWLNGKEYKCKVSNKALPAPEEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 10 - protein, 107 aa, synthetic construct [sequence omitted]

SEQ ID NO: 11 - protein, 451 aa, synthetic construct
EVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVQFNWYVDGVEVHNAKTKPREEQFNSTFRVVSVLTVVHQDWLNGKEYKCKVSNKALPAPEEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 12 - protein, 219 aa, synthetic construct
DIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 556 aa, Homo sapiens [sequence omitted]

(19) United States
(12) Patent Application Publication
(10) Pub. No.: US 2025/0074979 A1
(43) Pub. Date: Mar. 6, 2025

(54) ANTI-CD19 THERAPY IN COMBINATION WITH LENALIDOMIDE FOR THE TREATMENT OF LEUKEMIA OR LYMPHOMA

(71) Applicant: Incyte Corporation, Wilmington, DE (US)

(72) Inventors: Sumeet Ambarkhane, Munich (DE); Johannes Weirather, Oberhaching (DE)

(21) Appl. No.: 18/773,766

(22) Filed: Jul. 16, 2024

Related U.S. Application Data
(63) Continuation of application No. 17/084,751, filed on Oct. 30, 2020, now abandoned.

(30) Foreign Application Priority Data
Oct. 31, 2019 (EP) 19206463.2

(51) Int. Cl. C07K 16/28; A61K 31/454; A61K 39/00; A61K 39/395; A61P 35/00

(52) U.S. Cl. CPC C07K 16/2803; A61K 31/454; A61K 39/39558; A61P 35/00; A61K 2039/505; A61K 2039/545; C07K 2317/24; C07K 2317/51; C07K 2317/515; C07K 2317/52; C07K 2317/56; C07K 2317/565

(57) ABSTRACT

The present disclosure is directed to a therapeutic combination of an anti-CD19 antibody and lenalidomide for use in the treatment of hematological cancer patients. Furthermore, the present disclosure concerns extending the overall survival and/or the progression free survival in patients having specific types of hematological cancer.

This patent application is a continuation of U.S. application Ser. No. 17/084,751, filed Oct. 30, 2020, which claims the benefit of priority from EP 19206463.2 filed Oct. 31, 2019, teachings of each of which are herein incorporated by reference in their entirety.

### SEQUENCE LISTING

This application contains a Sequence Listing that has been submitted electronically as an XML file named “20443-0689002_SL_ST26.XML.” The XML file, created on Jul. 16, 2024, is 14,132 bytes in size. The material in the XML file is hereby incorporated by reference in its entirety.

### FIELD OF THE PRESENT DISCLOSURE

The present disclosure is directed to a therapeutic combination of an anti-CD19 antibody and lenalidomide for use in the treatment of hematological cancer patients. Furthermore, the present disclosure concerns extending the overall survival and/or the progression free survival in patients having specific types of hematological cancer.

### BACKGROUND

CD19 is a 95-kDa transmembrane glycoprotein of the immunoglobulin superfamily containing two extracellular immunoglobulin-like domains and an extensive cytoplasmic tail. The protein is a pan-B lymphocyte surface receptor and is ubiquitously expressed from the earliest stages of pre-B cell development onwards until it is down-regulated during terminal differentiation into plasma cells. It is B-lymphocyte lineage specific and not expressed on hematopoietic stem cells and other immune cells, except some follicular dendritic cells. CD19 functions as a positive regulator of B cell receptor (BCR) signalling and is important for B cell activation and proliferation and in the development of humoral immune responses. It acts as a co-stimulatory molecule in conjunction with CD21 and CD81 and is critical for B cell responses to T-cell-dependent antigens. The cytoplasmic tail of CD19 is physically associated with a family of tyrosine kinases that trigger downstream signalling pathways via the src-family of protein tyrosine kinases. CD19 is an attractive target for cancers of lymphoid origin since it is highly expressed in nearly all-chronic lymphocytic leukemia (CLL) and non-Hodgkin's lymphomas (NHL), as well as many other different types of leukemias, including acute lymphocytic leukemia (ALL) and hairy cell leukemia (HCL).

Tafasitamab (former names: MOR00208 and XmAb@5574) is a humanized monoclonal antibody that targets the antigen CD19, a transmembrane protein involved in B-cell receptor signalling. Tafasitamab has been engineered in the IgG Fc-region to enhance antibody-dependent cell-mediated cytotoxicity (ADCC), thus improving a key mechanism for tumor cell killing and offering potential for enhanced efficacy compared to conventional antibodies, i.e. non-enhanced antibodies. Tafasitamab has or is currently being studied in several clinical trials, such as in CLL, ALL and NHL. In some of those trials, Tafasitamab is used in combination with Idelalisib, Bendamustine or Venetoclax.

In the phase 2 L-MIND study (NCT02399085), the efficacy of Tafasitamab in combination with lenalidomide (LEN) is evaluated in adult patients with Relapsed or Refractory Diffuse Large B-cell Lymphoma (rr-DLBCL). L-MIND enrolled 81 patients with DLBCL ineligible for ASCT, who relapsed after or were refractory to 1-3 systemic regimens. Patients received co-administered Tafasitamab (12 mg/kg) and lenalidomide (25 mg/day) for up to 12 cycles (28-days each), followed by MOR00208 monotherapy (in patients with stable disease or better) until disease progression. The primary endpoint was objective response rate (centrally assessed). In this population of patients with relapsed or refractory DLBCL ineligible for stem cell transplant, combination treatment with Tafasitamab and lenalidomide elicited an overall objective response in 60% of patients and a complete response in 42.5% and indicates the combination of Tafasitamab and lenalidomide a promising treatment option.

The present disclosure concerns extending progression free survival in Relapsed or Refractory Diffuse Large B-cell Lymphoma (rr-DLBCL) cancer patient population; combining Tafasitamab and lenalidomide to treat patients pre-treated at least with one line or two lines of therapies (for example with R-CHOP (Rituximab and cyclophosphamide, adriamycin, vincristine and prednisone (CHOP)); extending the overall survival in Relapsed or Refractory Diffuse Large B-cell Lymphoma (rr-DLBCL) cancer patients; combining Tafasitamab and lenalidomide to treat patients having Relapsed or Refractory Diffuse Large B-cell Lymphoma (rr-DLBCL) wherein the Relapsed or Refractory Diffuse Large B-cell Lymphoma (rr-DLBCL) is a germinal center B-cell type (GCB) rr-DLBCL or a non-germinal center B-cell type (non-GCB) rr-DLBCL.

### SUMMARY OF PRESENT DISCLOSURE

The present disclosure provides a new treatment regimen for specific hematologic cancer patients. In particular, the present disclosure concerns the treatment of rr-DLBCL with a combination of an anti-CD19 antibody and lenalidomide.

In a first aspect, the present disclosure concerns a pharmaceutical composition comprising a therapeutic combination of an anti-CD19 antibody and lenalidomide for use in the treatment of hematological cancer patients wherein said treatment extends the overall survival and/or the progression free survival of said patients. In a another aspect, the present disclosure concerns a pharmaceutical composition comprising an anti-CD19 antibody for use in the treatment of hematological cancer patients wherein said anti-CD19 antibody is administered in combination with lenalidomide and wherein said treatment extends the overall survival and/or the progression free survival of said patients. In a another aspect, the present disclosure concerns a pharmaceutical composition comprising lenalidomide for use in the treatment of hematological cancer patients wherein lenalidomide is administered in combination with an anti-CD19 antibody and wherein said treatment extends the overall survival and/or the progression free survival of said patients.

In another aspect, the present disclosure concerns a method for extending progression free survival in a hematological cancer patient population comprising administering an anti-CD19 antibody and lenalidomide to the patients in the population.

In another aspect, the present disclosure concerns a therapeutic combination of an anti-CD19 antibody and lenalidomide for use in the treatment of a hematological cancer patient population wherein said treatment results in an extended overall survival in the patients in the population.

In a further aspect, the present disclosure concerns a therapeutic combination of an anti-CD19 antibody and lenalidomide for use in the treatment of a hematological cancer patient wherein said treatment results in an extended overall survival in said patient.

In a further aspect, the present disclosure concerns a therapeutic combination of an anti-CD19 antibody and lenalidomide for use in the treatment of a hematological cancer patient wherein said treatment results in an extended progression free survival in said patient.

In an embodiment the anti-CD19 antibody for use in the treatment of a hematological cancer patient in a therapeutic combination with lenalidomide comprises an HCDR1 region comprising the sequence SYVMH (SEQ ID NO: 1), an HCDR2 region comprising the sequence NPYNDG (SEQ ID NO: 2), an HCDR3 region comprising the sequence GTYYYGTRVFDY (SEQ ID NO: 3), an LCDR1 region comprising the sequence RSSKSLQNVNGNTYLY (SEQ ID NO: 4), an LCDR2 region comprising the sequence RMSNLNS (SEQ ID NO: 5), and an LCDR3 region comprising the sequence MQHLEYPIT (SEQ ID NO: 6).

In a further embodiment the anti-CD19 antibody for use in the treatment of a hematological cancer patient in combination with lenalidomide comprises a variable heavy chain of the sequence

(SEQ ID NO: 7)
EVQLVESGGGLVKPGGSLKLSCAASGYTFTSYVMHWVRQAPGKGLEWIGY
INPYNDGTKYNEKFQGRVTISSDKSISTAYMELSSLRSEDTAMYYCARGT
YYYGTRVFDYWGQGTLVTVSS

and a variable light chain of the sequence

(SEQ ID NO: 8)
DIVMTQSPATLSLSPGERATLSCRSSKSLQNVNGNTYLYWFQQKPGQSPQ
LLIYRMSNLNSGVPDRFSGSGSGTEFTLTISSLEPEDFAVYYCMQHLEYP
ITFGAGTKLEIK.

In another embodiment of the present disclosure the anti-CD19 antibody is a human, humanized or chimeric antibody. In another embodiment of the present disclosure the anti-CD19 antibody is of the IgG isotype. In another embodiment the antibody is IgG1, IgG2 or IgG1/IgG2 chimeric. In another embodiment of the present disclosure the isotype of the anti-CD19 antibody is engineered to enhance antibody-dependent cell-mediated cytotoxicity. In another embodiment the heavy chain constant region of the anti-CD19 antibody comprises amino acids 239D and 332E, wherein the Fc numbering is according to the EU index as in Kabat. In another embodiment the antibody is IgG1, IgG2 or IgG1/IgG2 and the chimeric heavy chain constant region of the anti-CD19 antibody comprises amino acids 239D and 332E, wherein the Fc numbering is according to the EU index as in Kabat.+

In a further embodiment the anti-CD19 antibody for use in the treatment of a hematological cancer patient in combination with lenalidomide comprises a heavy chain having the sequence

(SEQ ID NO: 11)
EVQLVESGGGLVKPGGSLKLSCAASGYTFTSYVMHWVRQAPGKGLEWIGY
INPYNDGTKYNEKFQGRVTISSDKSISTAYMELSSLRSEDTAMYYCARGT
YYYGTRVFDYWGQGTLVTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKKVEPKSCDKTHTCPPCPAPELLGGPDVFLFPPK
PKDTLMISRTPEVTCVVVDVSHEDPEVQFNWYVDGVEVHNAKTKPREEQF
NSTFRVVSVLTVVHQDWLNGKEYKCKVSNKALPAPEEKTISKTKGQPREP
QVYTLPPSREEMTKNQVSLTCLVKGFYPSDIAVEWESNGQPENNYKTTPP
MLDSDGSFFLYSKLTVDKSRWQQGNVFSCSVMHEALHNHYTQKSLSLSPG
K

and a light chain having the sequence

(SEQ ID NO: 12)
DIVMTQSPATLSLSPGERATLSCRSSKSLQNVNGNTYLYWFQQKPGQSPQ
LLIYRMSNLNSGVPDRFSGSGSGTEFTLTISSLEPEDFAVYYCMQHLEYP
ITFGAGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC

Optionally the therapeutic combination of an anti-CD19 antibody and lenalidomide results in a 12-months overall survival rate of 80% or more in the patients in the hematological cancer patient population. In one embodiment, the patients in the population to be treated have received one line of previous treatment. In another embodiment the one line of previous treatment was treatment with Rituximab. In one embodiment the one line of previous treatment was treatment with R-CHOP.

Optionally the therapeutic combination of an anti-CD19 antibody and lenalidomide results in a 12-months overall survival rate of 55% or more in the patients in the hematological cancer patient population. In one embodiment, the patients in the population to be treated have received two or more lines of previous treatment. In another embodiment the two or more lines of previous treatment included treatment with Rituximab. In another embodiment the two or more lines of previous treatment included treatment with R-CHOP.

Optionally the therapeutic combination of an anti-CD19 antibody and lenalidomide results in a 12-months overall survival rate of 60% or more in the patients in the hematological cancer patient population. In one embodiment, the patients in the population to be treated have a germinal center B-cell type (GCB) DLBCL.

Optionally the therapeutic combination of an anti-CD19 antibody and lenalidomide results in a 12-months overall survival rate of 80% or more in the patients in the hematological cancer patient population. In one embodiment, the patients in the population to be treated have a non-germinal center B-cell type (non-GCB) DLBCL.

The present disclosure provides a new treatment regimen for hematologic cancer. In particular, the present disclosure concerns the treatment of rr-DLBCL in human subjects with a combination of an anti-CD19 antibody and lenalidomide.

### BRIEF DESCRIPTION OF THE DRAWINGS

FIG. 1: Objective Response Rate by Baseline Characteristics

Two patients had double or triple hit DLBCL status—unknown at study entry. A single patient had ‘double hit’ DLBCL and achieved a best objective response of partial response. A single patient had ‘triple hit’ DLBCL and achieved a best objective response of complete response. (CI, confidence interval; DLBCL, diffuse large B-cell lymphoma; IHC, immunohistochemistry; IPI, International Prognostic Index; GCB, germinal center B-cell; LDH, lactate dehydrogenase).

FIG. 2: Swimmer plot of progression-free survival for patients with diffuse large B-cell lymphoma arising from transformation of low-grade lymphoma and double- or triple-hit lymphoma.

CR, complete response; DHL, double-hit lymphoma; IRC, independent review committee; PR, partial response; SD, stable disease; THL, triple-hit lymphoma; TL, transformed low-grade lymphoma.

### Definitions

The term “CD19” refers to the protein known as CD19, having the following synonyms: B4, B-lymphocyte antigen CD19, B-lymphocyte surface antigen B4, CVID3, Differentiation antigen CD19, MGC12802, and T-cell surface antigen Leu-12.

Human CD19 has the amino acid sequence of:

(SEQ ID NO: 13)
MPPPRLLFFLLFLTPMEVRPEEPLVVKVEEGDNAVLQCLKGTSDGPTQQL
TWSRESPLKPFLKLSLGLPGLGIHMRPLAIWLFIFNVSQQMGGFYLCQPG
PPSEKAWQPGWTVNVEGSGELFRWNVSDLGGLGCGLKNRSSEGPSSPSGK
LMSPKLYVWAKDRPEIWEGEPPCLPPRDSLNQSLSQDLTMAPGSTLWLSC
GVPPDSVSRGPLSWTHVHPKGPKSLLSLELKDDRPARDMWVMETGLLLPR
ATAQDAGKYYCHRGNLTMSFHLEITARPVLWHWLLRTGGWKVSAVTLAYL
IFCLCSLVGILHLQRALVLRRKRKRMTDPTRRFFKVTPPPGSGPQNQYGN
VLSLPTPTSGLGRAQRWAAGLGGTAPSYGNPSSDVQADGALGSRSPPGVG
PEEEEGEGYEEPDSEEDSEFYENDSNLGQDQLSQDGSGYENPEDEPLGPE
DEDSFSNAESYENEDEELTQPVARTMDFLSPHGSAWDPSREATSLGSQSY
EDMRGILYAAPQLRSIRGQPGPNHEEDADSYENMDNPDGPDPAWGGGGRM
GTWSTR

Tafasitamab”, “MOR00208” and “XmAb5574” are used as synonyms to describe the antibody of Table 1. Table 1 provides the amino acid sequences of Tafasitamab. Tafasitamab is described in U.S. patent application Ser. No. 12/377,251, which is incorporated by reference in its entirety. U.S. patent application Ser. No. 12/377,251 describes the antibody named 4G7 H1.52 Hybrid S239D/I332E/4G7 L1.155 (later named MOR00208 and Tafasitamab).

The term “antibody” as used herein refers to a protein comprising at least two heavy (H) chains and two light (L) chains inter-connected by disulfide bonds, which interacts with an antigen. Each heavy chain is comprised of a variable heavy chain region (abbreviated herein as VH) and a heavy chain constant region. The heavy chain constant region is comprised of three domains, CH1, CH2 and CH3. Each light chain is comprised of a variable light chain region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FR's arranged from amino-terminus to carboxy-terminus in the following order: FR1, CDR1, FR2, CDR2, FR3, CDR3, and FR4. The variable regions of the heavy and light chains contain a binding domain that interacts with an antigen. The term “antibody” includes for example, monoclonal antibodies, human antibodies, humanized antibodies, camelised antibodies and chimeric antibodies. The antibodies can be of any isotype (e.g., IgG, IgE, IgM, IgD, IgA and IgY), class (e.g., IgG1, IgG2, IgG3, IgG4, IgA1 and IgA2) or subclass. Both the light and heavy chains are divided into regions of structural and functional homology.

The phrase “antibody fragment”, as used herein, refers to one or more portions of an antibody that retain the ability to specifically interact with (e.g., by binding, steric hindrance, stabilizing spatial distribution) an antigen. Examples of binding fragments include, but are not limited to, a Fab fragment, a monovalent fragment consisting of the VL, VH, CL and CH1 domains; a F(ab)2 fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody; a dAb fragment (Ward et al., (1989) Nature 341:544-546), which consists of a VH domain; and an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al., (1988) Science 242:423-426; and Huston et al., (1988) Proc. Natl. Acad. Sci. 85:5879-5883). Such single chain antibodies are also intended to be encompassed within the term “antibody fragment”. These antibody fragments are obtained using conventional techniques known to those of skill in the art, and the fragments are screened for utility in the same manner as are intact antibodies. Antibody fragments can also be incorporated into single domain antibodies, maxibodies, minibodies, intrabodies, diabodies, triabodies, tetrabodies, v-NAR and bis-scFv (see, e.g., Hollinger and Hudson, (2005) Nature Biotechnology 23:1126-1136). Antibody fragments can be grafted into scaffolds based on polypeptides such as Fibronectin type III (Fn3) (see U.S. Pat. No. 6,703,199, which describes fibronectin polypeptide monobodies). Antibody fragments can be incorporated into single chain molecules comprising a pair of tandem Fv segments (VH-CH1-VH-CH1) which, together with complementary light chain polypeptides, form a pair of antigen-binding sites (Zapata et al., (1995) Protein Eng. 8:1057-1062; and U.S. Pat. No. 5,641,870).

“Administered” or “administration” includes but is not limited to delivery of a drug by an injectable form, such as, for example, an intravenous, intramuscular, intradermal or subcutaneous route or mucosal route, for example, as a nasal spray or aerosol for inhalation or as an ingestible solution, capsule or tablet. Preferably, the administration is by an injectable form.

The term “effector function” refers to those biological activities attributable to the Fc region of an antibody, which vary with the antibody isotype. Non-limiting examples of antibody effector functions include C1q binding and complement dependent cytotoxicity (CDC); Fc receptor binding and antibody-dependent cell-mediated cytotoxicity (ADCC) and/or antibody-dependent cellular phagocytosis (ADCP); down regulation of cell surface receptors (e.g. B cell receptor); and B cell activation.

“Antibody-dependent cell-mediated cytotoxicity” or “ADCC” refers to a form of cytotoxicity in which antibodies bound onto Fc receptors (FcRs) present on certain cytotoxic cells (e.g. NK cells, neutrophils, and macrophages) enable these cytotoxic effector cells to bind specifically to an antigen-bearing target cell and subsequently kill the target cell with cytotoxins. The primary cells for mediating ADCC, NK cells, express Fc□RIII only, whereas monocytes express FcyRI, FcyRII, and FcyRIII.

The term “hematologic cancer” includes blood-borne tumors and diseases or disorders involving abnormal cell growth and/or proliferation in tissues of hematopoietic origin, such as lymphomas, leukemias, and myelomas.

Non-Hodgkin's lymphoma (“NHL”) is a heterogeneous malignancy originating from lymphocytes. In the United States (U.S.), the incidence is estimated at 65,000/year with mortality of approximately 20,000 (American Cancer Society, 2006; and SEER Cancer Statistics Review). The disease can occur in all ages, the usual onset begins in adults over 40 years, with the incidence increasing with age. NHL is characterized by a clonal proliferation of lymphocytes that accumulate in the lymph nodes, blood, bone marrow and spleen, although any major organ may be involved. The current classification system used by pathologists and clinicians is the World Health Organization (WHO) Classification of Tumours, which organizes NHL into precursor and mature B-cell or T-cell neoplasms. The PDQ is currently dividing NHL as indolent or aggressive for entry into clinical trials. The indolent NHL group is comprised primarily of follicular subtypes, small lymphocytic lymphoma, MALT (mucosa-associated lymphoid tissue), and marginal zone; indolent encompasses approximately 50% of newly diagnosed B-cell NHL patients. Aggressive NHL includes patients with histologic diagnoses of primarily diffuse large B cell (DLBL, “DLBCL”, or DLCL) (40% of all newly diagnosed patients have diffuse large cell), Burkitt's, and mantle cell (“MCL”). The clinical course of NHL is highly variable. A major determinant of clinical course is the histologic subtype. Most indolent types of NHL are considered to be incurable disease. Patients respond initially to either chemotherapy or antibody therapy and most will relapse. Studies to date have not demonstrated an improvement in survival with early intervention. In asymptomatic patients, it is acceptable to “watch and wait” until the patient becomes symptomatic or the disease pace appears to be accelerating. Over time, the disease may transform to a more aggressive histology. The median survival is 8 to 10 years, and indolent patients often receive 3 or more treatments during the treatment phase of their disease. Initial treatment of the symptomatic indolent NHL patient historically has been combination chemotherapy. The most commonly used agents include: vincristine and prednisone (CVP); or cyclophosphamide, adriamycin, vincristine, prednisone (CHOP). Approximately 70% to 80% of patients will respond to their initial chemotherapy, duration of remissions last on the order of 2-3 years. Ultimately the majority of patients relapse. The discovery and clinical use of the anti-CD20 antibody, rituximab, has provided significant improvements in response and survival rate. The current standard of care for most patients is rituximab+CHOP (R-CHOP) or rituximab+CVP (R-CVP). Rituximab therapy has been shown to be efficacious in several types of NHL, and is currently approved as a first line treatment for both indolent (follicular lymphoma) and aggressive NHL (diffuse large B cell lymphoma). However, there are significant limitations of anti-CD20 monoclonal antibody (mAb), including primary resistance (50% response in relapsed indolent patients), acquired resistance (50% response rate upon re-treatment), rare complete response (2% complete resonse rate in relapsed population), and a continued pattern of relapse. Finally, many B cells do not express CD20, and thus many B-cell disorders are not treatable using anti-CD20 antibody therapy.

In addition to NHL there are several types of leukemias that result from dysregulation of B cells. Chronic lymphocytic leukemia (also known as “chronic lymphoid leukemia” or “CLL”), is a type of adult leukemia caused by an abnormal accumulation of B lymphocytes. In CLL, the malignant lymphocytes may look normal and mature, but they are not able to cope effectively with infection. CLL is the most common form of leukemia in adults. Men are twice as likely to develop CLL as women. However, the key risk factor is age. Over 75% of new cases are diagnosed in patients over age 50. More than 10,000 cases are diagnosed every year and the mortality is almost 5,000 a year (American Cancer Society, 2006; and SEER Cancer Statistics Review). CLL is an incurable disease but progresses slowly in most cases. Many people with CLL lead normal and active lives for many years. Because of its slow onset, early-stage CLL is generally not treated since it is believed that early CLL intervention does not improve survival time or quality of life. Instead, the condition is monitored over time. Initial CLL treatments vary depending on the exact diagnosis and the progression of the disease. There are dozens of agents used for CLL therapy. Combination chemotherapy regimens such as FCR (fludarabine, cyclophosphamide and rituximab), and BR (Ibrutinib and rituximab) are effective in both newly-diagnosed and relapsed CLL. Allogeneic bone marrow (stem cell) transplantation is rarely used as a first-line treatment for CLL due to its risk.

Another type of leukemia is Small lymphocytic lymphoma (“SLL”) that is considered a CLL variant that lacks the clonal lymphocytosis required for the CLL diagnosis, but otherwise shares pathological and immunophenotypic features (Campo et al., 2011). The definition of SLL requires the presence of lymphadenopathy and/or splenomegaly. Moreover, the number of B lymphocytes in the peripheral blood should not exceed 5×109/L. In SLL, the diagnosis should be confirmed by histopathologic evaluation of a lymph node biopsy whenever possible (Hallek et al., 2008). The incidence of SLL is approximately 25% of CLL in the US (Dores et al., 2007).

Another type of leukemia is acute lymphoblastic leukemia (ALL), also known as acute lymphocytic leukemia. ALL is characterized by the overproduction and continuous multiplication of malignant and immature white blood cells (also known as lymphoblasts) in the bone marrow. ‘Acute’ refers to the undifferentiated, immature state of the circulating lymphocytes (“blasts”), and that the disease progresses rapidly with life expectancy of weeks to months if left untreated. ALL is most common in childhood with a peak incidence of 4-5 years of age. Children of age 12-16 die more easily from it than others. Currently, at least 80% of childhood ALL are considered curable. Under 4,000 cases are diagnosed every year and the mortality is almost 1,500 a year (American Cancer Society, 2006; and SEER Cancer Statistics Review).

“Subject” or “patient” as used in this context refers to any mammal, including rodents, such as mouse or rat, and primates, such as cynomolgus monkey (Macaca fascicularis), rhesus monkey (Macaca mulatta) or humans (Homo sapiens). Preferably, the subject or patient is a primate, most preferably a human patient, even more preferably an adult human patient.

The “Fc region” is used to define the C-terminal region of an immunoglobulin heavy chain. The Fc region of an immunoglobulin generally comprises two constant domains, a CH2 domain and a CH3 domain. Unless otherwise specified herein, numbering of amino acid residues in the Fc region is according to the EU numbering system, also called the EU index, as described in Kabat et al., Sequences of Proteins of Immunological Interest, 5^{th} Ed. Public Health Service, National Institutes of Health, Bethesda, MD, 1991.

The agents which are administered according to the present disclosure are administered to the patient in a therapeutically effective amount. A “therapeutically effective amount” refers to an amount sufficient to provide some improvement of the clinical manifestations of a given disease or disorder. The amount that is effective for a particular therapeutic purpose will depend on the severity of the disease or injury as well as on the weight and general state of the subject. It will be understood that determination of an appropriate dosage may be achieved, using routine experimentation, by constructing a matrix of values and testing different points in the matrix, all of which is within the ordinary skills of a trained physician or clinical scientist.

“Survival” refers to the patient remaining alive, and includes overall survival as well as progression free survival.

“Overall survival” or “OS” refers to the patient remaining alive for a defined period of time, such as 12 months, 3 years, 5 years, etc from the time of diagnosis or treatment. For the purposes of the clinical trial described in the example, overall survival (OS) is defined as the time from the date of first dosing of the patient to the date of death from any cause.

“Progression free survival” or “PFS” refers to the patient remaining alive, without the cancer progressing or getting worse. For the purpose of the clinical trial described in the example, progression free survival (PFS) is defined as the time from the first dosing of a patient to the first documented progressive disease, or unmanageable toxicity, or death from any cause, whichever occurs first. Disease progression can be documented by any clinically accepted methods.

By “extending survival” or “improving surviving” is meant increasing overall survival or progression free survival in a patient treated in accordance with the present disclosure relative to an untreated patient and/or relative to a patient treated with one or more approved anti-tumor agents, but not receiving treatment in accordance with the present disclosure.

An “objective response” refers to a measurable response, including complete response (CR) or partial response (PR).

By “complete response” or “CR” is intended the disappearance of all signs of cancer in response to treatment. This does not always mean the cancer has been cured.

“Partial response” or “PR” refers to a decrease in the size of one or more tumors or lesions, or in the extent of cancer in the body, in response to treatment.

“Efficacy data” refers to the data obtained in controlled clinical trial showing that a drug effectively treats a disease, such as cancer. Efficacy data for MOR00208 is provided in the examples herein.

“In combination” refers to the administration of one therapy in addition to another therapy. As such, “in combination with” includes simultaneous (e.g., concurrent) and consecutive administration in any order. By way of non-limiting example, a first therapy (e.g., agent, such as an anti-CD19 antibody) may be administered before (e.g., 1 minute, 15 minutes, 30 minutes, 45 minutes, 1 hour, 2 hours, 3 hours, 4 hours, 5 hours, 6 hours, 7 hours, 8 hours, 12 hours, 24 hours, 48 hours, 72 hours, 96 hours, 1 week, 2 weeks, 3 weeks, 4 weeks, 5 weeks, 6 weeks, 8 weeks, 8 weeks, 9 weeks, 10 weeks, 11 weeks, or 12 weeks), concurrently, or after (e.g., 1 minute, 15 minutes, 30 minutes, 45 minutes, 1 hour, 2 hours, 3 hours, 4 hours, 5 hours, 6 hours, 7 hours, 8 hours, 12 hours, 24 hours, 48 hours, 72 hours, 96 hours, 1 week, 2 weeks, 3 weeks, 4 weeks, 5 weeks, 6 weeks, 7 weeks, 8 weeks, 9 weeks, 10 weeks, 11 weeks, or 12 weeks or longer) the administration of a second therapy (e.g., pharmaceutical agent or a pharmaceutically acceptable salt thereof) to a patient. In some embodiments, the term “combination” means that the anti-CD19 antibody and the pharmaceutical agent or a pharmaceutically acceptable salt thereof are administered simultaneously or consecutivley. In certain embodiments, the anti-CD19 antibody and the pharmaceutical agent or a pharmaceutically acceptable salt thereof are administered in separate compositions, i.e., wherein the anti-CD19 antibody and the pharmaceutical agent or a pharmaceutically acceptable salt thereof are administered each in a separate unit dosage form. It is understood that the anti-CD19 antibody and the pharmaceutical agent or a pharmaceutically acceptable salt thereof are administered on the same day or on different days and in any order as according to an appropriate dosing protocol.

A “thalidomide analog” includes, but is not limited to, thalidomide itself, lenalidomide (CC-5013, Revlimid™), Pomalidomide (CC4047, Actimid™) and the compounds disclosed in WO2002068414 and WO2005016326, which are incorporated by reference in their entireties. The term refers to a synthetic chemical compound using the thalidomide structure as a backbone (e.g., side groups have been added or such groups have been deleted from the parent structure). The analog differs in structure from thalidomide and its metabolite compounds such as by a difference in the length of an alkyl chain, a molecular fragment, by one or more functional groups, or change in ionization. The term “thalidomide analog” also includes the metabolites of thalidomide. Thalidomide analogs include the racemic mixture of the S- and the R-enantiomer of a respective compound and the S-enantiomer or to the R-enantiomer individually. The racemic mixture is preferred.

Thalidomide analogs include compounds such as lenalidomide which has the following structure:

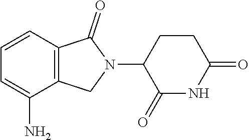 [SMILES: Nc1cccc2c1CN(C1CCC(=O)NC1=O)C2=O]

### DETAILED DESCRIPTION OF THE PRESENT DISCLOSURE AND EMBODIMENTS

In one aspect, the present disclosure concerns a method of treating rr-DLBCL in a human subject, comprising administering to the subject combination of an anti-CD19 antibody and lenalidomide.

The use of a CD19 antibody in non-specific B cell lymphomas is discussed in WO2007076950 (US2007154473), which are both incorporated by reference. The use of a CD19 antibody in CLL, NHL and ALL is described in Scheuermann et al., CD19 Antigen in Leukemia and Lymphoma Diagnosis and Immunotherapy, Leukemia and Lymphoma, Vol. 18, 385-397 (1995), which is incorporated by reference in its entirety.

Additional antibodies specific for CD19 are described in WO2005012493 (U.S. Pat. No. 7,109,304), WO2010053716 (U.S. Ser. No. 12/266,999) (Immunomedics); WO2007002223 (U.S. Pat. No. 8,097,703) (Medarex); WO2008022152 (Ser. No. 12/377,251) and WO2008150494 (Xencor), WO2008031056 (U.S. Ser. No. 11/852,106) (Medimmune); WO 2007076950 (U.S. Ser. No. 11/648,505) (Merck Patent GmbH); WO 2009/052431 (U.S. Ser. No. 12/253,895) (Seattle Genetics); and WO2010095031 (Ser. No. 12/710,442) (Glenmark Pharmaceuticals), WO2012010562 and WO2012010561 (International Drug Development), WO2011147834 (Roche Glycart), and WO 2012/156455 (Sanofi), which are all incorporated by reference in their entireties.

A pharmaceutical composition includes an active agent, e.g. an antibody for therapeutic use in humans. A pharmaceutical composition may additionally include pharmaceutically acceptable carriers or excipients.

In one aspect, the present disclosure concerns a method of treating rr-DLBCL in a human subject, comprising administering to the subject combination of an anti-CD19 antibody and lenalidomide, wherein the anti-CD19 antibody is administered at a dose of 12 mg/kg in all treatment cycles.

In another aspect, the present disclosure concerns a method of improving survival in a human subject with rr-DLBCL, comprising administering to the subject an anti-CD19 antibody and lenalidomide.

In yet another aspect, the present disclosure concerns an anti-CD19 antibody for use in the treatment of rr-DLBCL in a human subject in combination with lenalidomide.

In a further aspect, the present disclosure concerns the use of an anti-CD19 antibody in the preparation of a medicament for the treatment of rr-DLBCL, wherein the treatment comprises administration of the anti-CD19 antibody in combination with lenalidomide.

In a still further aspect, the present disclosure concerns the use of an anti-CD19 antibody in the preparation of a medicament for the treatment of rr-DLBCL, wherein the treatment comprises administration of an anti-CD19 antibody in combination with lenalidomide. In another aspect, the present disclosure concerns a kit comprising a container comprising an anti-CD19 antibody and instructions for administration of the anti-CD19 antibody to treat rr-DLBCL in a subject in combination with lenalidomide.

In yet another aspect, the present disclosure concerns a kit comprising a container comprising an anti-CD19 antibody and instructions for administration of an anti-CD19 antibody to treat rr-DLBCL in a subject in combination with lenalidomide.

In all aspects, in a particular embodiment the patient did receive prior anti-cancer treatment for hematological cancer. In all aspects, in particular embodiments, the administration of an anti-CD19 antibody in combination with lenalidomide improves survival, including overall survival (OS) and/or progression free survival (PFS) and/or response rate (RR).

In another embodiment, the present disclosure provides a therapeutic combination of an anti-CD19 antibody and lenalidomide for use in the treatment of hematological cancer patients wherein said hematological cancer patients have received one line of previous treatment and wherein the 12-months overall survival rate of said patients is extended to 60%, 70%, 80%, 83%, 85% or 87% or more. In a further embodiment the 12-months progression free survival of said patients is extended to 40%, 45%, 50%, 55%, 58% or more.

In another embodiment, the present disclosure provides a therapeutic combination of an anti-CD19 antibody and lenalidomide for use in the treatment of hematological cancer patients wherein said hematological cancer patients have received two or more lines of previous treatment and wherein the 12-months overall survival rate of said patients is extended to 40%, 45%, 50% 55% or more. In a further embodiment the 12-months progression free survival of said patients is extended to 30%, 35%, 40% or more.

In another embodiment, the present disclosure provides a therapeutic combination of an anti-CD19 antibody and lenalidomide for use in the treatment of hematological cancer patients wherein said hematological cancer patients have a germinal center B-cell type (GCB) DLBCL and wherein the 12-months overall survival rate of said patients is extended to 50%, 55%, 60% or 64% or more. In a further embodiment the 12-months progression free survival of said patients is extended to 30%, 35% or 37% or more.

In another embodiment, the present disclosure provides a therapeutic combination of an anti-CD19 antibody and lenalidomide for use in the treatment of hematological cancer patients wherein said hematological cancer patients have a non-germinal center B-cell type (non-GCB) DLBCL and wherein the 12-months overall survival rate of said patients is extended to 70%, 75%, 80% or 83% or more. In a further embodiment the 12-months progression free survival of said patients is extended to 60%, 65%, 70% or 73% or more.

In all aspects, the treatment comprises administration of an anti-CD19 antibody and lenalidomide for up to 12 cycles (28-days each). In one embodiment the treatment is followed by MOR00208 monotherapy until disease progression.

In all aspects, the treatment comprises administration of an anti-CD19 antibody and lenalidomide and the anti-CD19 antibody is administered intravenously at a dose of 12 mg/kg. In one embodiment said intravenous administration is over approximately 2 hours.

In some aspects the treatment comprises administration of an anti-CD19 antibody and lenalidomide for up to 12 cycles wherein for cycles 1 to 3, the anti-CD19 antibody is administered weekly on days 1, 8, 15, and 22. In one embodiment an additional loading dose of the anti-CD19 antibody is administered on day 4 of cycle 1. In another embodiment from cycle 4 onwards, the anti-CD19 antibody is administered every 14 days, on days 1 and 15 of each cycle.

In all aspects, the treatment comprises co-administration of an anti-CD19 antibody and lenalidomide and hematological cancer patients self-administered lenalidomide orally, starting with 25 mg daily on days 1-21 of each 28-day cycle. A step-wise dose reduction (decrease by 5 mg/day in each step, only once per cycle, without re-escalation) of lenalidomide was permitted in case of protocol-defined toxicities.

In a particular aspect, the present disclosure concerns a method of treating a hematological cancer patient by administering an anti-CD19 antibody and lenalidomide to the patient in an amount to improve progression free survival (PFS) and/or overall survival (OS), wherein said patient has received one line of previous treatment. In one embodiment the one line of previous treatment was treatment with R-CHOP. In another embodiment the one line of previous treatment included treatment with Rituximab. In one embodiment the anti-CD19antibody is tafasitamab. In one embodiment the 12-months progression free survival (PFS) rate is improved to more than 55%. In one embodiment the 12-months overall survival (OS) rate is improved to more than 85%. In one embodiment the 12-months progression free survival (PFS) rate is improved to more than 55% and the 12-months overall survival (OS) rate is improved to more than 85%. In one embodiment the amount of tafasitamab to improve progression free survival (PFS) and/or overall survival (OS) is 12 mg/kg per dose. In another embodiment the amount of tafasitamab to improve progression free survival (PFS) and/or overall survival (OS) is a regimen of up to 12 cycles wherein for cycles 1 to 3, tafasitamab is administered weekly on days 1, 8, 15, and 22 and from cycle 4 onwards, tafasitamab is administered every 14 days, on days 1 and 15 of each cycle. In one embodiment the amount of lenalidomide to improve progression free survival (PFS) and/or overall survival (OS) is 25 mg daily. In a further embodiment the amount of tafasitamab and lenalidomide to improve progression free survival (PFS) and/or overall survival (OS) is 12 mg/kg per dose of tafasitamab and 25 mg daily of lenalidomide. In another embodiment tafasitamab is administered in a regimen of up to 12 cycles wherein for cycles 1 to 3, tafasitamab is administered weekly on days 1, 8, 15, and 22 and from cycle 4 onwards, tafasitamab is administered every 14 days, on days 1 and 15 of each cycle and lenalidomide is administered daily on days 1-21 of each 28-day cycle with a step-wise dose reduction (decrease by 5 mg/day in each step, only once per cycle, without re-escalation) of lenalidomide in case of protocol-defined toxicities.

In a particular aspect, the present disclosure concerns a method of treating a hematological cancer patient by administering an anti-CD19 antibody and lenalidomide to the patient in an amount to improve progression free survival (PFS) and/or overall survival (OS), wherein said patient has received two or more lines of previous treatment. In one embodiment said patient received two lines of previous treatment. In one embodiment the two lines of previous treatment included treatment with R-CHOP. In another embodiment the two lines of previous treatment included treatment with Rituximab. In one embodiment the anti-CD19 antibody is tafasitamab. In one embodiment the 12-months progression free survival (PFS) rate is improved to more than 35%. In one embodiment the 12-months overall survival (OS) rate is improved to more than 55%. In one embodiment the 12-months progression free survival (PFS) rate is improved to more than 35% and the 12-months overall survival (OS) rate is improved to more than 55%. In one embodiment the amount of tafasitamab to improve progression free survival (PFS) and/or overall survival (OS) is 12 mg/kg per dose. In another embodiment the amount of tafasitamab to improve progression free survival (PFS) and/or overall survival (OS) is a regimen of up to 12 cycles wherein for cycles 1 to 3, tafasitamab is administered weekly on days 1, 8, 15, and 22 and from cycle 4 onwards, tafasitamab is administered every 14 days, on days 1 and 15 of each cycle. In one embodiment the amount of lenalidomide to improve progression free survival (PFS) and/or overall survival (OS) is 25 mg daily. In a further embodiment the amount of tafasitamab and lenalidomide to improve progression free survival (PFS) and/or overall survival (OS) is 12 mg/kg per dose of tafasitamab and 25 mg daily of lenalidomide. In another embodiment tafasitamab is administered in a regimen of up to 12 cycles wherein for cycles 1 to 3, tafasitamab is administered weekly on days 1, 8, 15, and 22 and from cycle 4 onwards, tafasitamab is administered every 14 days, on days 1 and 15 of each cycle and lenalidomide is administered daily on days 1-21 of each 28-day cycle with a step-wise dose reduction (decrease by 5 mg/day in each step, only once per cycle, without re-escalation) of lenalidomide in case of protocol-defined toxicities.

In a particular aspect, the present disclosure concerns a method of treating a hematological cancer patient by administering an anti-CD19 antibody and lenalidomide to the patient in an amount to improve 12-months progression free survival (PFS) and/or 12-months overall survival (OS), wherein said patient has a germinal center B-cell type (GCB) DLBCL. In one embodiment said patient has a germinal center B-cell type (GCB) rr-DLBCL. In one embodiment said patient has a germinal center B-cell type (GCB) rr-DLBCL and received at least one line of previous treatment wherein said previous treatment comprises treatment with R-CHOP. In one embodiment the anti-CD19 antibody is tafasitamab. In one embodiment the 12-months progression free survival (PFS) rate is improved to more than 35%. In one embodiment the 12-months overall survival (OS) rate is improved to more than 60%. In one embodiment the 12-months progression free survival (PFS) rate is improved to more than 35% and the 12-months overall survival (OS) rate is improved to more than 60%. In one embodiment the amount of tafasitamab to improve progression free survival (PFS) and/or overall survival (OS) is 12 mg/kg per dose. In another embodiment the amount of tafasitamab to improve progression free survival (PFS) and/or overall survival (OS) is a regimen of up to 12 cycles wherein for cycles 1 to 3, tafasitamab is administered weekly on days 1, 8, 15, and 22 and from cycle 4 onwards, tafasitamab is administered every 14 days, on days 1 and 15 of each cycle. In one embodiment the amount of lenalidomide to improve progression free survival (PFS) and/or overall survival (OS) is 25 mg daily. In a further embodiment the amount of tafasitamab and lenalidomide to improve progression free survival (PFS) and/or overall survival (OS) is 12 mg/kg per dose of tafasitamab and 25 mg daily of lenalidomide. In another embodiment tafasitamab is administered in a regimen of up to 12 cycles wherein for cycles 1 to 3, tafasitamab is administered weekly on days 1, 8, 15, and 22 and from cycle 4 onwards, tafasitamab is administered every 14 days, on days 1 and 15 of each cycle and lenalidomide is administered daily on days 1-21 of each 28-day cycle with a step-wise dose reduction (decrease by 5 mg/day in each step, only once per cycle, without re-escalation) of lenalidomide in case of protocol-defined toxicities.

In a particular aspect, the present disclosure concerns a method of treating a hematological cancer patient by administering an anti-CD19 antibody and lenalidomide to the patient in an amount to improve 12-months progression free survival (PFS) and/or 12-months overall survival (OS), wherein said patient has a non-germinal center B-cell type (non-GCB) DLBCL. In one embodiment said patient has a non-germinal center B-cell type (non-GCB) rr-DLBCL. In one embodiment said patient has a non-germinal center B-cell type (non-GCB) rr-DLBCL and received at least one line of previous treatment wherein said previous treatment comprises treatment with R-CHOP. In one embodiment the anti-CD19 antibody is tafasitamab. In one embodiment the 12-months progression free survival (PFS) rate is improved to more than 70%. In one embodiment the 12-months overall survival (OS) rate is improved to more than 80%. In one embodiment the 12-months progression free survival (PFS) rate is improved to more than 70% and the 12-months overall survival (OS) rate is improved to more than 80%. In one embodiment the amount of tafasitamab to improve progression free survival (PFS) and/or overall survival (OS) is 12 mg/kg per dose. In another embodiment the amount of tafasitamab to improve progression free survival (PFS) and/or overall survival (OS) is a regimen of up to 12 cycles wherein for cycles 1 to 3, tafasitamab is administered weekly on days 1, 8, 15, and 22 and from cycle 4 onwards, tafasitamab is administered every 14 days, on days 1 and 15 of each cycle. In one embodiment the amount of lenalidomide to improve progression free survival (PFS) and/or overall survival (OS) is 25 mg daily. In a further embodiment the amount of tafasitamab and lenalidomide to improve progression free survival (PFS) and/or overall survival (OS) is 12 mg/kg per dose of tafasitamab and 25 mg daily of lenalidomide. In another embodiment tafasitamab is administered in a regimen of up to 12 cycles wherein for cycles 1 to 3, tafasitamab is administered weekly on days 1, 8, 15, and 22 and from cycle 4 onwards, tafasitamab is administered every 14 days, on days 1 and 15 of each cycle and lenalidomide is administered daily on days 1-21 of each 28-day cycle with a step-wise dose reduction (decrease by 5 mg/day in each step, only once per cycle, without re-escalation) of lenalidomide in case of protocol-defined toxicities.

In another aspect, the present disclosure concerns a method of improving or extending survival in a human subject with rr-DLBCL, comprising administering to the subject an anti-CD19 antibody and lenalidomide.

In yet another aspect, the present disclosure concerns an anti-CD19 antibody for use in the treatment of rr-DLBCL in a human subject in combination with lenalidomide.

In a further aspect, the present disclosure concerns the use of an anti-CD19 antibody in the preparation of a medicament for the treatment of rr-DLBCL, wherein the treatment comprises administration of the anti-CD19 antibody in combination with lenalidomide.

In a still further aspect, the present disclosure concerns the use of an anti-CD19 antibody in the preparation of a medicament for the treatment of rr-DLBCL, wherein the treatment comprises administration of an anti-CD19 antibody in combination with lenalidomide. In another aspect, the present disclosure concerns a kit comprising a container comprising an anti-CD19 antibody and instructions for administration of the anti-CD19 antibody to treat rr-DLBCL in a subject in combination with lenalidomide.

In yet another aspect, the present disclosure concerns a kit comprising a container comprising an anti-CD19 antibody and instructions for administration of an anti-CD19 antibody to treat rr-DLBCL in a subject in combination with lenalidomide.

In all aspects, in a particular embodiment the patient did receive prior anti-cancer treatment for hematological cancer. In all aspects, in particular embodiments, the administration of an anti-CD19 antibody in combination with lenalidomide improves survival, including overall survival (OS) and/or progression free survival (PFS) and/or response rate (RR).

In all aspects, the treatment comprises administration of an anti-CD19 antibody and lenalidomide for up to 12 cycles (28-days each). In embodiments the treatment is followed by anti-CD19 antibody monotherapy until disease progression.

In all aspects, the treatment comprises administration of an anti-CD19 antibody and lenalidomide and the anti-CD19 antibody is administered intravenously at a dose of 12 mg/kg. In one embodiment said intravenous administration is over approximately 2 hours.

In some aspects the treatment comprises administration of an anti-CD19 antibody and lenalidomide for up to 12 cycles wherein for cycles 1 to 3, the anti-CD19 antibody is administered weekly on days 1, 8, 15, and 22. In one embodiment an additional loading dose of the anti-CD19 antibody is administered on day 4 of cycle 1. In another embodiment from cycle 4 onwards, the anti-CD19 antibody is administered every 14 days, on days 1 and 15 of each cycle.

In all aspects, the treatment comprises administration of an anti-CD19 antibody and lenalidomide and hematological cancer patients self-administered lenalidomide orally, starting with 25 mg daily on days 1-21 of each 28-day cycle. A step-wise dose reduction (decrease by 5 mg/day in each step, only once per cycle, without re-escalation) of lenalidomide was permitted in case of protocol-defined toxicities.

In an embodiment the anti-CD19 antibody for use in the treatment of hematological cancer patients in combination with lenalidomide comprises a variable heavy chain of the sequence

(SEQ ID NO: 7)
EVQLVESGGGLVKPGGSLKLSCAASGYTFTSYVMHWVRQAPGKGLEWIGY
INPYNDGTKYNEKFQGRVTISSDKSISTAYMELSSLRSEDTAMYYCARGT
YYYGTRVFDYWGQGTLVTVSS

and a variable light chain of the sequence

(SEQ ID NO: 8)
DIVMTQSPATLSLSPGERATLSCRSSKSLQNVNGNTYLYWFQQKPGQSPQ
LLIYRMSNLNSGVPDRFSGSGSGTEFTLTISSLEPEDFAVYYCMQHLEYP
ITFGAGTKLEIK

or a variable heavy chain and and a variable light chain that has at least 80%, at least 90%, at least 95%, at least 96%, at least 97%, at least 98% or at least 99% identity to the variable heavy chain of SEQ ID NO: 7 and to the variable light chain of SEQ ID NO: 8.

In an embodiment the anti-CD19 antibody for use in the treatment of hematological cancer patients in combination with lenalidomide comprises a variable heavy chain of the sequence

(SEQ ID NO: 7)
EVQLVESGGGLVKPGGSLKLSCAASGYTFTSYVMHWVRQAPGKGLEWIGY
INPYNDGTKYNEKFQGRVTISSDKSISTAYMELSSLRSEDTAMYYCARGT
YYYGTRVFDYWGQGTLVTVSS

and a variable light chain of the sequence

(SEQ ID NO: 8)
DIVMTQSPATLSLSPGERATLSCRSSKSLQNVNGNTYLYWFQQKPGQSPQ
LLIYRMSNLNSGVPDRFSGSGSGTEFTLTISSLEPEDFAVYYCMQHLEYP
ITFGAGTKLEIK

or a variable heavy chain and and a variable light chain that has at least 80%, at least 90%, at least 95%, at least 96%, at least 97%, at least 98% or at least 99% identity to the variable heavy chain of SEQ ID NO: 7 and to the variable light chain of SEQ ID NO: 8, wherein the anti-CD19 antibody comprises an HCDR1 region comprising the sequence SYVMH (SEQ ID NO: 1), an HCDR2 region comprising the sequence NPYNDG (SEQ ID NO: 2), an HCDR3 region comprising the sequence GTYYYGTRVFDY (SEQ ID NO: 3), an LCDR1 region comprising the sequence RSSKSLQNVNGNTYLY (SEQ ID NO: 4), an LCDR2 region comprising the sequence RMSNLNS (SEQ ID NO: 5), and an LCDR3 region comprising the sequence MQHLEYPIT (SEQ ID NO: 6). In another embodiment the heavy chain region of the anti-CD19 antibody comprises amino acids 239D and 332E, wherein the Fc numbering is according to the EU index as in Kabat.

In a further embodiment the anti-CD19 antibody for use in the treatment of hematological cancer patients in combination with lenalidomide comprises a heavy chain having the sequence

(SEQ ID NO: 11)
EVQLVESGGGLVKPGGSLKLSCAASGYTFTSYVMHWVRQAPGKGLEWIGY
INPYNDGTKYNEKFQGRVTISSDKSISTAYMELSSLRSEDTAMYYCARGT
YYYGTRVFDYWGQGTLVTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKKVEPKSCDKTHTCPPCPAPELLGGPDVFLFPPK
PKDTLMISRTPEVTCVVVDVSHEDPEVQFNWYVDGVEVHNAKTKPREEQF
NSTFRVVSVLTVVHQDWLNGKEYKCKVSNKALPAPEEKTISKTKGQPREP
QVYTLPPSREEMTKNQVSLTCLVKGFYPSDIAVEWESNGQPENNYKTTPP
MLDSDGSFFLYSKLTVDKSRWQQGNVFSCSVMHEALHNHYTQKSLSLSPG
K

and a light chain having the sequence

(SEQ ID NO: 12)
DIQMTQSPATLSLSPGERATLSCRSSKSLQNVNGNTYLYWFQQKPGQSPQ
LLIYRMSNLNSGVPDRFSGSGSGTEFTLTISSLEPEDFAVYYCMQHLEYP
ITFGAGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC

or a heavy chain and and a light chain that has at least 80%, at least 90%, at least 95%, at least 96%, at least 97%, at least 98% or at least 99% identity to the heavy chain of SEQ ID NO: 7 and to the light chain of SEQ ID NO: 8.

In a further embodiment the anti-CD19 antibody for use in the treatment of hematological cancer patients in combination with lenalidomide comprises a heavy chain having the sequence

(SEQ ID NO: 11)
EVQLVESGGGLVKPGGSLKLSCAASGYTFTSYVMHWVRQAPGKGLEWIGY
INPYNDGTKYNEKFQGRVTISSDKSISTAYMELSSLRSEDTAMYYCARGT
YYYGTRVFDYWGQGTLVTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKKVEPKSCDKTHTCPPCPAPELLGGPDVFLFPPK
PKDTLMISRTPEVTCVVVDVSHEDPEVQFNWYVDGVEVHNAKTKPREEQF
NSTFRVVSVLTVVHQDWLNGKEYKCKVSNKALPAPEEKTISKTKGQPREP
QVYTLPPSREEMTKNQVSLTCLVKGFYPSDIAVEWESNGQPENNYKTTPP
MLDSDGSFFLYSKLTVDKSRWQQGNVFSCSVMHEALHNHYTQKSLSLSPG
K

and a light chain having the sequence

(SEQ ID NO: 12)
DIVMTQSPATLSLSPGERATLSCRSSKSLQNVNGNTYLYWFQQKPGQSPQ
LLIYRMSNLNSGVPDRFSGSGSGTEFTLTISSLEPEDFAVYYCMQHLEYP
ITFGAGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC

or a heavy chain and and a light chain that has at least 80%, at least 90%, at least 95%, at least 96%, at least 97%, at least 98% or at least 99% identity to the heavy chain of SEQ ID NO: 7 and to the light chain of SEQ ID NO: 8 and wherein the anti-CD19 antibody comprises an HCDR1 region comprising the sequence SYVMH (SEQ ID NO: 1), an HCDR2 region comprising the sequence NPYNDG (SEQ ID NO: 2), an HCDR3 region comprising the sequence GTYYYGTRVFDY (SEQ ID NO: 3), an LCDR1 region comprising the sequence RSSKSLQNVNGNTYLY (SEQ ID NO: 4), an LCDR2 region comprising the sequence RMSNLNS (SEQ ID NO: 5), and an LCDR3 region comprising the sequence MQHLEYPIT (SEQ ID NO: 6). In another embodiment the heavy chain region of the anti-CD19 antibody comprises amino acids 239D and 332E, wherein the Fc numbering is according to the EU index as in Kabat.

In other embodiments the present disclosure refers to an anti-CD19 antibody for use in the treatment of hematological cancer patients in combination with lenalidomide wherein said patients have received one, at least one, two or at least two lines of previous treatment and wherein after the treatment with an anti-CD19 antibody in combination with lenalidomide said patients have
(i) a progression-free survival (PFS) of at least 8 months, at least 9 months, at least 10 months, at least 11 months, at least 12 months, at least 13 months, at least 14 months, at least 15 months, at least 16 months, at least 17 months, at least 18 months, at least 19 months, at least 20 months, at least 24 months, at least 30 months, at least 36 months, at least 42 months, at least 48 months or at least 54 months;
(ii) an objective response rate (ORR) of at least 30%, at least 40%, at least 50%, at least 60%, at least 70%, at least 80% or at least 80%;
(iii) a duration of response (DoR) over at least at least 10 months, at least 12 months, at at least 14 months, at least 16 months, at least 18 months, at least 20 months, at least 24 months, at least 30 months, at least 36 months, at least 42 months, at least 48 months or at least 54 months;
(iv) an overall survival (OS) of at least at least 10 months, at least 12 months, at at least 14 months, at least 16 months, at least 18 months, at least 20 months, at least 24 months, at least 30 months, at least 36 months, at least 42 months, at least 48 months or at least 54 months; or
(v) a combination of one or more of the foregoing.

In another embodiment of the present disclosure said anti-CD19 antibody is administered in combination with lenalidomide in a dosing regimen as disclosed herein.

In other embodiments the present disclosure refers to an anti-CD19 antibody for the treatment of hematological cancer patients in combination with lenalidomide wherein said patients have a germinal center B-cell type (GCB) DLBCL and wherein said patients after said treatment have
(i) a progression-free survival (PFS) of at least 8 months, at least 9 months, at least 10 months, at least 11 months, at least 12 months, at least 13 months, at least 14 months, at least 15 months, at least 16 months, at least 17 months, at least 18 months, at least 19 months, at least 20 months, at least 24 months, at least 30 months, at least 36 months, at least 42 months, at least 48 months or at least 54 months;
(ii) an objective response rate (ORR) of at least 30%, at least 40%, at least 50%, at least 60%, at least 70%, at least 80% or at least 80%;
(iii) a duration of response (DoR) over at least at least 10 months, at least 12 months, at at least 14 months, at least 16 months, at least 18 months, at least 20 months, at least 24 months, at least 30 months, at least 36 months, at least 42 months, at least 48 months or at least 54 months;
(iv) an overall survival (OS) of at least at least 10 months, at least 12 months, at at least 14 months, at least 16 months, at least 18 months, at least 20 months, at least 24 months, at least 30 months, at least 36 months, at least 42 months, at least 48 months or at least 54 months; or
(vi) a combination of one or more of the foregoing.

In another embodiment of the present disclosure said anti-CD19 antibody is administered in combination with lenalidomide in a dosing regimen as disclosed herein.

In other embodiments the present disclosure refers to an anti-CD19 antibody for the treatment of hematological cancer patients in combination with lenalidomide wherein said patients have a non-germinal center B-cell type (non-GCB) DLBCL and wherein said patients after said treatment have
(i) a progression-free survival (PFS) of at least 8 months, at least 9 months, at least 10 months, at least 11 months, at least 12 months, at least 13 months, at least 14 months, at least 15 months, at least 16 months, at least 17 months, at least 18 months, at least 19 months, at least 20 months, at least 24 months, at least 30 months, at least 36 months, at least 42 months, at least 48 months or at least 54 months;
(ii) an objective response rate (ORR) of at least 30%, at least 40%, at least 50%, at least 60%, at least 70%, at least 80% or at least 80%;
(iii) a duration of response (DoR) over at least at least 10 months, at least 12 months, at at least 14 months, at least 16 months, at least 18 months, at least 20 months, at least 24 months, at least 30 months, at least 36 months, at least 42 months, at least 48 months or at least 54 months;
(iv) an overall survival (OS) of at least at least 10 months, at least 12 months, at at least 14 months, at least 16 months, at least 18 months, at least 20 months, at least 24 months, at least 30 months, at least 36 months, at least 42 months, at least 48 months or at least 54 months or
(vii) a combination of one or more of the foregoing.

In another embodiment of the present disclosure said anti-CD19 antibody is administered in combination with lenalidomide in a dosing regimen as disclosed herein.

In other embodiments the present disclosure refers to an anti-CD19 antibody for the treatment of hematological cancer patients in combination with lenalidomide wherein said combination treatment extends one or more of the following features:
(i) the progression-free survival (PFS),
(ii) the objective response rate (ORR),
(iii) the duration of response (DoR),
(iv) the overall survival (OS),
(v) the time to progression (TTP).

In another embodiment said one or more of the features (i) to (v) are extended relative to the treatment comprising an anti-CD20 antibody. In a further embodiment said one or more of the features (i) to (v) are extended in comparison to the treatment comprising an anti-CD20 antibody and a chemotherapeutic. In a further embodiment said anti-CD20 antibody is rituximab or a biosimilar thereof. In further embodiments said one or more of the features (i) to (v) are extended in comparison to the treatment comprising an anti-CD20 antibody and one or more of cyclophosphamide, adriamycin, vincristine or prednisone. In a further embodiment said one or more of the features (i) to (v) are extended in comparison to the treatment comprising R-CHOP.

In other embodiments the present disclosure refers to a therapeutic combination comprising an anti-CD19 antibody and lenalidomide for use in the treatment of hematological cancer patients wherein said patients have received one, at least one, two, or at least two lines of previous treatment and wherein the administration of said anti-CD19 antibody results in extended progression-free survival (PFS), improved objective response rate (ORR), improved duration of response (DoR), extended overall survival (OS) or extended time to progression (TTP).

In other embodiments the present disclosure refers to a therapeutic combination comprising an anti-CD19 antibody and lenalidomide for use in the treatment of hematological cancer patients wherein said patients have received one, at least one, two, or at least two lines of previous treatment and wherein the administration of said anti-CD19 antibody results in improved progression-free survival (PFS) relative to the administration of an anti-CD20 antibody, improved objective response rate (ORR) relative to the administration of an anti-CD20 antibody, improved duration of response (DoR) relative to the administration of an anti-CD20 antibody, improved overall survival (OS) relative to the administration of an anti-CD20 antibody or improved time to progression (TTP) relative to the administration of an anti-CD20 antibody.

In other embodiments the present disclosure refers to a therapeutic combination comprising an anti-CD19 antibody and lenalidomide for use in the treatment of hematological cancer patients wherein said patients have a germinal center B-cell type (GCB) DLBCL and wherein the administration of said anti-CD19 antibody results in extended progression-free survival (PFS), improved objective response rate (ORR), improved duration of response (DoR), improved overall survival (OS) or improved time to progression (TTP).

In other embodiments the present disclosure refers to a therapeutic combination comprising an anti-CD19 antibody and lenalidomide for use in the treatment of hematological cancer patients wherein said patients have a germinal center B-cell type (GCB) DLBCL and wherein the administration of said anti-CD19 antibody results in improved progression-free survival (PFS) relative to the administration of an anti-CD20 antibody, improved objective response rate (ORR) relative to the administration of an anti-CD20 antibody, improved duration of response (DoR) relative to the administration of an anti-CD20 antibody, improved overall survival (OS) relative to the administration of an anti-CD20 antibody or improved time to progression (TTP) relative to the administration of an anti-CD20 antibody.

In other embodiments the present disclosure refers to a therapeutic combination comprising an anti-CD19 antibody and lenalidomide for use in the treatment of hematological cancer patients wherein said patients have a non-germinal center B-cell type (non-GCB) DLBCL and wherein the administration of said anti-CD19 antibody results in extended progression-free survival (PFS), improved objective response rate (ORR), improved duration of response (DoR), improved overall survival (OS) or improved time to progression (TTP).

In other embodiments the present disclosure refers to a therapeutic combination comprising an anti-CD19 antibody and lenalidomide for use in the treatment of hematological cancer patients wherein said patients have a non-germinal center B-cell type (non-GCB) DLBCL and wherein the administration of said anti-CD19 antibody results in improved progression-free survival (PFS) relative to the administration of an anti-CD20 antibody, improved objective response rate (ORR) relative to the administration of an anti-CD20 antibody, improved duration of response (DoR) relative to the administration of an anti-CD20 antibody, improved overall survival (OS) relative to the administration of an anti-CD20 antibody or improved time to progression (TTP) relative to the administration of an anti-CD20 antibody.

In other embodiments the present disclosure refers to an anti-CD19 antibody for use in the treatment of hematological cancer patients in combination with lenalidomide wherein the administration of said anti-CD19 antibody in combination with lenalidomide results in extended progression-free survival (PFS) relative to the administration of R-CHOP, extended objective response rate (ORR) relative to the administration of R-CHOP, extended duration of response (DoR) relative to the administration of R-CHOP, extended overall survival (OS) relative to the administration of R-CHOP or extended time to progression (TTP) relative to the administration of R-CHOP.

In another embodiment said hematologic cancer patient has double hit diffuse large B cell lymphoma.

In another embodiment said hematologic cancer patient has triple hit diffuse large B cell lymphoma.

In another embodiment said hematologic cancer patient has double hit or triple hit diffuse large B cell lymphoma.

In another embodiment said hematologic cancer patient is a hematologic cancer patient with diffuse large B-cell lymphoma arising from transformation of low-grade lymphoma.

In one embodiment the present disclosure provides an anti-CD19 antibody wherein said anti-CD19 antibody is administered in a concentration of 12 mg/kg.

In a further embodiment, the anti-CD19 antibody is administered weekly, bi-weekly or monthly. In a further embodiment the anti-CD19 antibody is administered weekly for the first 3 months and bi-weekly for at least the next 3 months. In a further embodiment, the anti-CD19 antibody is administered weekly for the first 3 months. In a further embodiment the anti-CD19 antibody is administered weekly for the first 3 months and bi-weekly for at least the next 3 months. In another embodiment the anti-CD19 antibody is administered weekly for the first 3 months, bi-weekly for the next 3 months and monthly thereafter. In yet another embodiment the anti-CD19 antibody is administered weekly for the first 3 months, bi-weekly for the next 3 months and monthly thereafter.

### Indications and Patients

The present disclosure provides a therapeutic combination comprising an anti-CD19 antibody and lenalidomide for use in the treatment of a hematological cancer patient wherein said hematologic cancer patient has chronic lymphocytic leukemia (CLL), non-Hodgkin's lymphoma (NHL), small lymphocytic lymphoma (SLL) or acute lymphoblastic leukemia (ALL). In another embodiment said hematologic cancer patient has non-Hodgkin's lymphoma. In further embodiments the non-Hodgkin's lymphoma is selected from the group consisting of follicular lymphoma, small lymphocytic lymphoma, mucosa-associated lymphoid tissue, marginal zone lymphoma, diffuse large B cell lymphoma, Burkitt's lymphoma and mantle cell lymphoma. In further embodiments the non-Hodgkin's lymphoma is Relapsed or Refractory Diffuse Large B-cell Lymphoma (rr-DLBCL). In another embodiment said hematologic cancer patient has diffuse large B cell lymphoma and is not eligible for High-Dose Chemotherapy (HDC) and/or Autologous Stem-Cell Transplantation (ASCT). In another embodiment said hematologic cancer patient has Relapsed or Refractory Diffuse Large B-cell Lymphoma (rr-DLBCL) and is not eligible for High-Dose Chemotherapy (HDC) and/or Autologous Stem-Cell Transplantation (ASCT). In another embodiment said hematologic cancer patient has Relapsed or Refractory Diffuse Large B-cell Lymphoma (rr-DLBCL) arising from low grade lymphoma and is not eligible for High-Dose Chemotherapy (HDC) and/or Autologous Stem-Cell Transplantation (ASCT). In further embodiments the non-Hodgkin's lymphoma is Relapsed or Refractory Diffuse Large B-cell Lymphoma (rr-DLBCL) arising from low grade lymphoma.

In another embodiment said hematologic cancer patient has diffuse large B cell lymphoma wherein the patient is selected based on one or more of the following criteria:
1. Age>18 years
2. Histologically confirmed diagnosis of DLBCL
3. Tumour tissue for central pathology review and correlative studies must be provided.
4. Patients must have:
   a. relapsed and/or refractory disease
   b at least one bidimensionally measurable, PET positive disease site (transverse diameter of ≥1.5 cm and perpendicular diameter of ≥1.0 cm at baseline)
   c. received at least one, but no more than three previous systemic regimens for the treatment of DLBCL and one therapy line must have included a CD20-targeted therapy
   d. Eastern Cooperative Oncology Group 0 to 2
5. Patients not considered in the opinion of the investigator eligible, or patients unwilling to undergo intensive salvage therapy including ASCT
6. Patients must meet the following laboratory criteria at screening:
   a. absolute neutrophil count≥1.5×109/L
   b. platelet count≥90×109/L
   c. total serum bilirubin≤2.5×ULN or ≤5×ULN in cases of Glibert's Syndrome or liver involvement by lymphoma
   d. alanine transaminase, aspartate aminotransferase and alkaline phosphatase≤3×ULN or <5×ULN in cases of liver involvement
   e. serum creatinine clearance≥60 mL/minute
7. Females of childbearing potential (FCBP) must:
   a. not be pregnant
   b. refrain from breastfeeding and donating blood or oocytes
   c. agree to ongoing pregnancy testing
   d. commit to continued abstinence from heterosexual intercourse, or agree to use and be able to comply with the use of double-barrier contraception
8. Males (if sexually active with a FCBP) must
   a. use an effective barrier method of contraception
   b. refrain from donating blood or sperm
9. In the opinion of the investigator the patients must:
   a. be able and willing to receive adequate prophylaxis and/or therapy for thromboembolic events
   b. be able to understand the reason for complying with the special conditions of the pregnancy prevention risk management plan and give written acknowledgement of this.

In another embodiment said hematologic cancer patient has double hit diffuse large B cell lymphoma.

In another embodiment said hematologic cancer patient has triple hit diffuse large B cell lymphoma.

In another embodiment said hematologic cancer patient has double hit or triple hit diffuse large B cell lymphoma.

In another embodiment said hematologic cancer patient is a hematologic cancer patient with diffuse large B-cell lymphoma arising from transformation of low-grade lymphoma.

In another embodiment said hematologic cancer patient has diffuse large B cell lymphoma wherein the patient is excluded based on one or more of the following exclusion criteria:
1. Patients who have:
   a. other histological type of lymphoma
   b. primary refractory DLBCL
   c. a history of “double/triple hit” genetics
2. Patients who have, within 14 days prior to Day 1 dosing:
   a. not discontinued CD20-targeted therapy, chemotherapy, radiotherapy, investigational anticancer therapy or other lymphoma specific therapy
   b. undergone major surgery or suffered from significant traumatic injury
   c. received live vaccines.
   d. required parenteral antimicrobial therapy for active, intercurrent infections
3. Patients who:
   a. were previously treated with CD19-targeted therapy or IMiDs® (e.g. thalidomide, LEN)
   b. have undergone ASCT within the period ≤3 months prior to signing the informed consent form.
   c. have undergone previous allogenic stem cell transplantation
   d. have a history of deep venous thrombosis/embolism and who are not willing/able to take venous thromboembolic event prophylaxis during the entire treatment period
   e. concurrently use other anticancer or experimental treatments
4. Prior history of malignancies other than DLBCL, unless the patient has been free of the disease for ≥5 years prior to screening.
5. Patients with:
   a. positive hepatitis B and/or C serology.
   b. known seropositivity for or history of active viral infection with human immunodeficiency virus (HIV)
   c. CNS lymphoma involvement
   d. history or evidence of clinically significant cardiovascular, CNS and/or other systemic disease that would in the investigator's opinion preclude participation in the study or compromise the patient's ability to give informed consent.

### Method

In another embodiment of the present disclosure the predicted benefit from the therapeutic administration of an anti-CD19 antibody in combination with lenalidomide is improved progression-free survival (PFS), improved objective response rate (ORR), improved duration of response (DoR), improved overall survival (OS) or improved time to progression (TTP) or a combination thereof.

In another embodiment of the present disclosure the predicted benefit from the therapeutic administration of an anti-CD19 antibody in combination with lenalidomide is improved progression-free survival (PFS) relative to the administration of an anti-CD20 antibody, improved objective response rate (ORR) relative to the administration of an anti-CD20 antibody, improved duration of response (DoR) relative to the administration of an anti-CD20 antibody, improved overall survival (OS) relative to the administration of an anti-CD20 antibody or improved time to progression (TTP) relative to the administration of an anti-CD20 antibody or a combination thereof.

In another embodiment of the present disclosure the predicted benefit from the therapeutic administration of an anti-CD19 antibody in combination with lenalidomide is
(i) a progression-free survival (PFS) of at least 8 months, at least 9 months, at least 10 months, at least 11 months, at least 12 months, at least 13 months, at least 14 months, at least 15 months, at least 16 months, at least 17 months, at least 18 months, at least 19 months or at least 20 months;
(ii) an objective response rate (ORR) of at least 30%, at least 40%, at least 50%, at least 60%, at least 70%, at least 80% or at least 80%;
(iii) a duration of response (DoR) over at least at least 10 months, at least 12 months, at at least 14 months, at least 16 months, at least 18 months, at least 20 months, at least 24 months, at least 30 months, at least 36 months, at least 42 months, at least 48 months or at least 54 months;
(iv) an overall survival (OS) of at least at least 10 months, at least 12 months, at at least 14 months, at least 16 months, at least 18 months, at least 20 months, at least 24 months, at least 30 months, at least 36 months, at least 42 months, at least 48months or at least 54 months; or
(v) a combination of one or more of the foregoing.

In another embodiment of the present disclosure said anti-CD19 antibody is administered in combination with a pharmaceutical agent as disclosed herein.

In another embodiment of the present disclosure the predicted benefit from the therapeutic administration of an anti-CD19 antibody in combination with lenalidomide is improved progression-free survival (PFS) relative to the administration of an anti-CD20 antibody and a chemotherapeutic, improved objective response rate (ORR) relative to the administration of an anti-CD20 antibody and a chemotherapeutic, improved duration of response (DoR) relative to the administration of an anti-CD20 antibody and a chemotherapeutic, improved overall survival (OS) relative to the administration of an anti-CD20 antibody and a chemotherapeutic or improved time to progression (TTP) relative to the administration of an anti-CD20 antibody and a chemotherapeutic. In a further embodiment said anti-CD20 antibody is rituximab or a biosimilar thereof. In further embodiments said chemotherapeutic comprises one or more of cyclophosphamide, adriamycin, vincristine or prednisone.

In another embodiment of the present disclosure the predicted benefit from the therapeutic administration of an anti-CD19 antibody in combination with lenalidomide is improved progression-free survival (PFS) relative to the administration of R-CHOP, improved objective response rate (ORR) relative to the administration of R-CHOP, improved duration of response (DoR) relative to the administration of R-CHOP, improved overall survival (OS) relative to the administration of R-CHOP or improved time to progression (TTP) relative to the administration of R-CHOP.

In another embodiment of the present disclosure said predicted benefit from the therapeutic administration of an anti-CD19 antibody in combination with lenalidomide is an increase of one or more of the following features:
(i) the progression-free survival (PFS),
(ii) the objective response rate (ORR),
(iii) the duration of response (DoR),
(iv) the overall survival (OS),
(v) the time to progression (TTP).

In another embodiment said increase of one or more of the features (i) to (v) are in comparison to the treatment comprising an anti-CD20 antibody. In a further embodiment said increase of one or more of the features (i) to (v) are in comparison to the treatment comprising an anti-CD20 antibody and a chemotherapeutic. In a further embodiment said anti-CD20 antibody is rituximab or a biosimilar thereof. In a further embodiment said increase of one or more of the features (i) to (v) are in comparison to the treatment comprising an anti-CD20 antibody and one or more of cyclophosphamide, adriamycin, vincristine or prednisone. In a further embodiment said increase of one or more of the features (i) to (v) are in comparison to the treatment comprising R-CHOP.

In an embodiment of the present disclosure said hematologic cancer patient who is predicted to benefit from the therapeutic administration of an anti-CD19 antibody and lenalidomide has chronic lymphocytic leukemia (CLL), non-Hodgkin's lymphoma (NHL), small lymphocytic lymphoma (SLL) or acute lymphoblastic leukemia (ALL). In a further embodiment said hematologic cancer patient has non-Hodgkin's lymphoma. In a further embodiment said hematologic cancer patient has non-Hodgkin's lymphoma, wherein the non-Hodgkin's lymphoma is selected from the group consisting of follicular lymphoma, small lymphocytic lymphoma, mucosa-associated lymphoid tissue, marginal zone lymphoma, diffuse large B cell lymphoma, Burkitt's lymphoma and mantle cell lymphoma. In a further embodiment said hematologic cancer patient has Relapsed or Refractory Diffuse Large B-cell Lymphoma (rr-DLBCL). In a further embodiment said hematologic cancer patient has Relapsed or Refractory Diffuse Large B-cell Lymphoma (rr-DLBCL) and received one, at least one, two, or at least two lines of previous treatment. In a further embodiment said hematologic cancer patient has Relapsed or Refractory Diffuse Large B-cell Lymphoma (rr-DLBCL) and received one line of previous treatment. In a further embodiment said hematologic cancer patient has Relapsed or Refractory Diffuse Large B-cell Lymphoma (rr-DLBCL) and received Rituximab as a previous treatment. In a further embodiment said hematologic cancer patient has Relapsed or Refractory Diffuse Large B-cell Lymphoma (rr-DLBCL) and received R-CHOP as a previous treatment. In a further embodiment said hematologic cancer patient has Relapsed or Refractory Diffuse Large B-cell Lymphoma (rr-DLBCL) and received two lines of previous treatment. In a further embodiment said hematologic cancer patient has Relapsed or Refractory Diffuse Large B-cell Lymphoma (rr-DLBCL) wherein the patient has a non-germinal center B-cell type (non-GCB) DLBCL. In a further embodiment said hematologic cancer patient has Relapsed or Refractory Diffuse Large B-cell Lymphoma (rr-DLBCL) wherein the patient has a germinal center B-cell type (GCB) DLBCL.

In a further embodiment of the present disclosure said hematologic cancer patient who is predicted to benefit from the therapeutic administration of an anti-CD19 antibody and lenalidomide is administered with an anti-CD19 antibody that comprises an HCDR1 region comprising the sequence SYVMH (SEQ ID NO: 1), an HCDR2 region comprising the sequence NPYNDG (SEQ ID NO: 2), an HCDR3 region comprising the sequence GTYYYGTRVFDY (SEQ ID NO: 3), an LCDR1 region comprising the sequence RSSKSLQNVNGNTYLY (SEQ ID NO: 4), an LCDR2 region comprising the sequence RMSNLNS (SEQ ID NO: 5), and an LCDR3region comprising the sequence MQHLEYPIT (SEQ ID NO: 6). In another embodiment said anti-CD19 antibody comprises a variable heavy chain of the sequence

(SEQ ID NO: 7)
EVQLVESGGGLVKPGGSLKLSCAASGYTFTSYVMHWVRQAPGKGLEWIGY
INPYNDGTKYNEKFQGRVTISSDKSISTAYMELSSLRSEDTAMYYCARGT
YYYGTRVFDYWGQGTLVTVSS

and a variable light chain of the sequence

(SEQ ID NO: 8)
DIVMTQSPATLSLSPGERATLSCRSSKSLQNVNGNTYLYWFQQKPGQSPQ
LLIYRMSNLNSGVPDRFSGSGSGTEFTLTISSLEPEDFAVYYCMQHLEYP
ITFGAGTKLEIK.

In a further embodiment said anti-CD19 antibody comprises a heavy chain having the sequence

(SEQ ID NO: 11)
EVQLVESGGGLVKPGGSLKLSCAASGYTFTSYVMHWVRQAPGKGLEWIGY
INPYNDGTKYNEKFQGRVTISSDKSISTAYMELSSLRSEDTAMYYCARGT
YYYGTRVFDYWGQGTLVTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKKVEPKSCDKTHTCPPCPAPELLGGPDVFLFPPK
PKDTLMISRTPEVTCVVVDVSHEDPEVQFNWYVDGVEVHNAKTKPREEQF
NSTFRVVSVLTVVHQDWLNGKEYKCKVSNKALPAPEEKTISKTKGQPREP
QVYTLPPSREEMTKNQVSLTCLVKGFYPSDIAVEWESNGQPENNYKTTPP
MLDSDGSFFLYSKLTVDKSRWQQGNVFSCSVMHEALHNHYTQKSLSLSPG
K

and a light chain having the sequence

(SEQ ID NO: 12)
DIVMTQSPATLSLSPGERATLSCRSSKSLQNVNGNTYLYWFQQKPGQSPQ
LLIYRMSNLNSGVPDRFSGSGSGTEFTLTISSLEPEDFAVYYCMQHLEYP
ITFGAGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC.

In another aspect, the present disclosure concerns a method of treatment of a hematologic cancer patient having rr-DLBCL, comprising administering to the subject an anti-CD19 antibody and lenalidomide.

In yet another aspect, the present disclosure concerns an anti-CD19 antibody for use in the treatment of a hematologic cancer patient having rr-DLBCL in combination with lenalidomide.

In a further aspect, the present disclosure concerns the use of an anti-CD19 antibody in the preparation of a medicament for the treatment of a hematologic cancer patient having rr-DLBCL, wherein the treatment comprises administration of the anti-CD19 antibody in combination with lenalidomide.

In another embodiment said hematologic cancer patient has double hit diffuse large B cell lymphoma.

In another embodiment said hematologic cancer patient has triple hit diffuse large B cell lymphoma.

In another embodiment said hematologic cancer patient has double hit or triple hit diffuse large B cell lymphoma.

In another embodiment said hematologic cancer patient is a hematologic cancer patient with diffuse large B-cell lymphoma arising from transformation of low-grade lymphoma. In another embodiment said hematologic cancer patient is a hematologic cancer patient with diffuse large B-cell lymphoma arising from transformation of low-grade lymphoma, wherein said low-grade lymphoma includes but is not limited to follicular lymphoma or marginal zone lymphoma. In some embodiments said diffuse large B-cell lymphoma arising from transformation of low-grade lymphoma is transformed follicular lymphoma or transformed marginal zone lymphoma. In another embodiment said hematologic cancer patient is a hematologic cancer patient with diffuse large B-cell lymphoma wherein such diffuse large B-cell lymphoma is a transformed lymphoma. In another embodiment said hematologic cancer patient is a hematologic cancer patient with diffuse large B-cell lymphoma wherein such diffuse large B-cell lymphoma is a transformed indolent lymphoma. In another embodiment said hematologic cancer patient is a hematologic cancer patient with diffuse large B-cell lymphoma wherein such diffuse large B-cell lymphoma has transformed from a low-grade lymphoma or an indolent lymphoma. In further embodiments the hematologic cancer patient with diffuse large B-cell lymphoma has Relapsed or Refractory Diffuse Large B-cell Lymphoma (rr-DLBCL). In another embodiment said hematologic cancer patient has Relapsed or Refractory diffuse large B cell lymphoma and is not eligible for High-Dose Chemotherapy (HDC) and/or Autologous Stem-Cell Transplantation (ASCT). In another embodiment said hematologic cancer patient has Relapsed or Refractory Diffuse Large B-cell Lymphoma (rr-DLBCL) and is not eligible for High-Dose Chemotherapy (HDC) and/or Autologous Stem-Cell Transplantation (ASCT). In another embodiment said hematologic cancer patient has Relapsed or Refractory Diffuse Large B-cell Lymphoma (rr-DLBCL) arising from low grade lymphoma and is not eligible for High-Dose Chemotherapy (HDC) and/or Autologous Stem-Cell Transplantation (ASCT). In further embodiments the non-Hodgkin's lymphoma is Relapsed or Refractory Diffuse Large B-cell Lymphoma (rr-DLBCL) arising from low grade lymphoma.

### Antibody Sequences

TABLE 1

| | SEQ ID NO: | Amino Acids |
|---|---|---|
| HCDR1 | SEQ ID NO: 1 | SYVMH |
| HCDR2 | SEQ ID NO: 2 | NPYNDG |
| HCDR3 | SEQ ID NO: 3 | GTYYYGTRVFDY |
| LCDR1 | SEQ ID NO: 4 | RSSKSLQNVNGNTYLY |
| LCDR2 | SEQ ID NO: 5 | RMSNLNS |
| LCDR3 | SEQ ID NO: 6 | MQHLEYPIT |
| VH | SEQ ID NO: 7 | EVQLVESGGGLVKPGGSLKLSCAASGYTFTSYVMH |
| | | WVRQAPGKGLEWIGYINPYNDGTKYNEKFQGRVTIS |
| | | SDKSISTAYMELSSLRSEDTAMYYCARGTYYYGTRV |
| | | FDYWG QGTLVTVSS |
| VL | SEQ ID NO: 8 | DIVMTQSPATLSLSPGERATLSCRSSKSLQNVNGNT |
| | | YLYWFQQKPGQSPQLLIYRMSNLNSGVPDRFSGSG |
| | | SGTEFTLTISSLEPEDFAVYYCMQHLEYPITFGAGTKL |
| | | EIK |
| Heavy chain constant | SEQ ID NO: 9 | ASTKGPSVFPLAPSSKSTSGGTAALGCLVKDYFPEP |
| domain | | VTVSWNSGALTSGVHTFPAVLQSSGLYSLSSVVTVP |
| | | SSSLGTQTYICNVNHKPSNTKVDKKVEPKSCDKTHT |
| | | CPPCPAPELLGGPDVFLFPPKPKDTLMISRTPEVTCV |
| | | VVDVSHEDPEVQFNWYVDGVEVHNAKTKPREEQFN |
| | | STFRVVSVLTVVHQDWLNGKEYKCKVSNKALPAPEE |
| | | KTISKTKGQPREPQVYTLPPSREEMTKNQVSLTCLVK |
| | | GFYPSDIAVEWESNGQPENNYKTTPPMLDSDGSFFL |
| | | YSKLTVDKSRWQQGNVFSCSVMHEALHNHYTQKSL |
| | | SLSPGK |
| Light chain constant | SEQ ID NO: 10 | RTVAAPSVFIFPPSDEQLKSGTASVVCLLNNFYPREA |
| domain | | KVQWKVDNALQSGNSQESVTEQDSKD |
| | | STYSLSSTLTLSKADYEKHKVYACEVTHQGLSSPVTK |
| | | SFNRGEC |
| Full Heavy chain | SEQ ID NO: 11 | EVQLVESGGGLVKPGGSLKLSCAASGYTFTSYVMH |
| | | WVRQAPGKGLEWIGYINPYNDGTKYNEKFQGRVTIS |
| | | SDKSISTAYMELSSLRSEDTAMYYCARGTYYYGTRV |
| | | FDYWGQGTLVTVSSASTKGPSVFPLAPSSKSTSGGT |
| | | AALGCLVKDYFPEPVTVSWNSGALTSGVHTFPAVLQ |
| | | SSGLYSLSSVVTVPSSSLGTQTYICNVNHKPSNTKVD |
| | | KKVEPKSCDKTHTCPPCPAPELLGGPDVFLFPPKPK |
| | | DTLMISRTPEVTCVVVDVSHEDPEVQFNWYVDGVEV |
| | | HNAKTKPREEQFNSTFRVVSVLTVVHQDWLNGKEY |
| | | KCKVSNKALPAPEEKTISKTKGQPREPQVYTLPPSRE |
| | | EMTKNQVSLTCLVKGFYPSDIAVEWESNGQPENNYK |
| | | TTPPMLDSDGSFFLYSKLTVDKSRWQQGNVFSCSV |
| | | MHEALHNHYTQKSLSLSPGK |
| Full Light chain | SEQ ID NO: 12 | DIVMTQSPATLSLSPGERATLSCRSSKSLQNVNGNT |
| | | YLYWFQQKPGQSPQLLIYRMSNLNSGVPDRFSGSG |
| | | SGTEFTLTISSLEPEDFAVYYCMQHLEYPITFGAGTKL |
| | | EIKRTVAAPSVFIFPPSDEQLKSGTASVVCLLNNFYPR |
| | | EAKVQWKVDNALQSGNSQESVTEQDSKDSTYSLSS |
| | | TLTLSKADYEKHKVYACEVTHQGLSSPVTKSFNRGE |
| | | C |

### EXAMPLES

### Example 1: MOR00208 Plus Lenalidomide in Relapsed or Refractory Diffuse Large B-Cell Lymphoma

Patients with relapsed or refractory diffuse large B-cell lymphoma (DLBCL) typically have poor outcomes and limited treatment options. MOR00208 is an Fc-enhanced humanized anti-CD19 monoclonal antibody that showed preclinical activity and single-agent activity in patients with relapsed or refractory B-cell malignancies. Treatment of relapsed or refractory diffuse large B-cell lymphoma (DLBCL) patients with MOR00208 in combination with lenalidomide was clinically investigated.

### Patients and Study Design

This open-label, single-arm, multicenter, phase 2 study commenced in March 2016. Patients were enrolled until November 2017 at 35 sites (Supplementary Appendix) in 10 countries across Europe and the USA. Adult patients (over 18 years old) with histologically confirmed DLBCL (including indolent lymphoma with a subsequent DLBCL relapse), and who relapsed after or were refractory to at least one, but no more than three systemic regimens (with at least one anti-CD20 therapy) and who were not candidates for high-dose chemotherapy and subsequent ASCT, were eligible. Additional inclusion criteria were adequate organ function, Eastern Cooperative Oncology Group performance status of 0 to 2, and measurable disease at baseline. Exclusion criteria included any other histological type of lymphoma, a history of ‘double/triple hit’ DLBCL if already known, prior treatment with anti-CD19 therapy or immunomodulatory drugs such as thalidomide or lenalidomide, or primary refractory DLBCL, defined as no response to or progression during or within 6 months of frontline therapy. Prior to a protocol amendment, only patients who relapsed within 3 months of a prior anti-CD20-containing regimen were defined as primary refractory and excluded. Thus, patients having relapsed or progressed between 3 and 6 months of frontline therapy were recruited before the protocol amendment, and considered primary refractory patients.

Treatment comprised co-administration of MOR00208 and lenalidomide for up to 12 cycles (28-days each), followed by MOR00208 monotherapy (in patients with stable disease or better) until disease progression. MOR00208 was administered intravenously at a dose of 12 mg/kg, over approximately 2 hours. For cycles 1 to 3, MOR00208 was administered weekly on days 1, 8, 15, and 22; an additional loading dose was administered on day 4 of cycle 1.From cycle 4, MOR00208 was administered every 14 days,22 on days 1 and 15 of each cycle. Patients self-administered lenalidomide orally, starting with 25 mg daily on days 1-21 of each 28-day cycle. A step-wise dose reduction (decrease by 5 mg/day in each step, only once per cycle, without re-escalation) of lenalidomide was permitted in case of protocol-defined toxicities.

The primary endpoint was objective response rate, defined as complete response plus partial response. Secondary endpoints included disease control rate (complete plus partial response plus stable disease), duration of response, time to next treatment, progression-free survival, overall survival, time to progression, incidence and severity of adverse events, as well as immunogenicity (presence of anti-MOR00208 antibodies), pharmacokinetics, and biomarker analyses (including B-, T-, and NK-cell measurements over time, cell of origin).

Analysis of the primary endpoint occurred when all patients had completed a minimum of 12 months of follow-up. Efficacy analyses are based on the full analysis set comprising all patients who received at least one dose of both MOR00208 and lenalidomide; safety analyses were based on those who received at least one dose of either study medication. Sample size was determined assuming that combination treatment could improve the objective response rate from 20% (monotherapy) to 35% (combination therapy). Applying an exact binomial test with a two-sided significance level of 5% and a power of 85%, the estimated sample size was 73 patients. Assuming a drop-out rate of 10%, a total sample size of 80 patients was estimated. Statistical analysis was performed using SAS® Software version 9.4 or above (SAS Institute, Cary, N.C.).

### Results

A total of 81 patients were enrolled and received at least one dose of either study medication (and evaluated for safety), and 80 received at least one dose of both MOR00208 and lenalidomide (evaluated for efficacy). In total, 30 (37.0%) patients successfully completed 12 cycles of MOR00208 and lenalidomide therapy and 28 (34.6%) were receiving MOR00208 monotherapy at data cut-off.

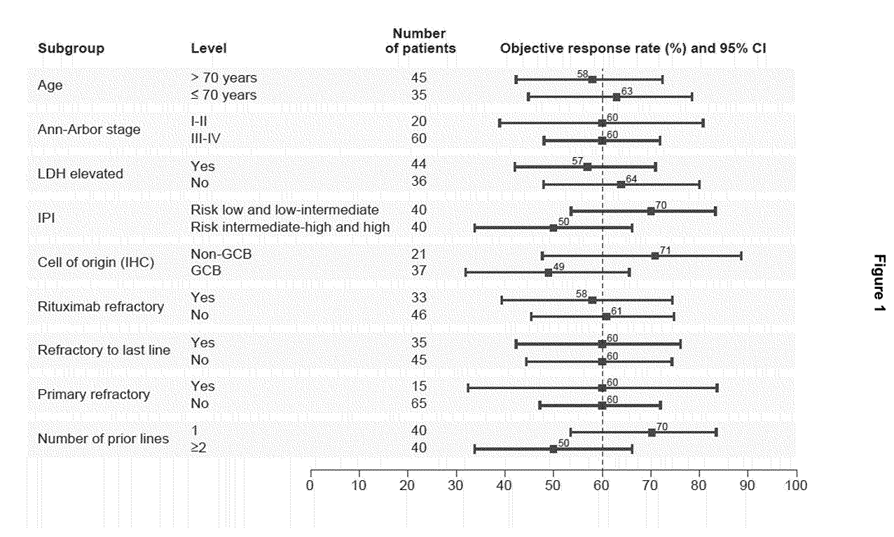

The assessed objective response rate was 60.0% (95% confidence interval [CI], 48.4 to 70.8%), with 34 (42.5%) patients achieving a complete response and 14 (17.5%) achieving a partial response (Table 2). The overall concordance between both centrally and investigator-assessed objective response rate was 88.2%. Positron emission tomography scans performed in 30/34 (88.2%) patients with a complete response confirmed computerized tomography-derived results in all cases. Disease control rate was 73.8% (95% CI, 62.7 to 83.0% in 59 patients). The median time to response (partial or complete response) was 2 months (range 1.7 to 16.8 months), and median time to complete response was 7.1 months (range 1.7 to 17.0 months). Analysis of objective response rate by patient baseline characteristics indicated high and consistent response rates across most subgroups (FIG. 1), including those refractory to prior therapies.

TABLE 2
Best Objective Response (Per Independent
Radiology/Clinical Review Committee).

| | MOR00208 plus |
|---|---|
| Best objective response, n (%) | lenalidomide (N = 80) |
| CR | 34 (42.5%) |
| PR | 14 (17.5%) |
| SD | 11 (13.8%) |
| PD | 13 (16.3%) |
| NE* | 8 (10.0%) |
| PET-confirmed CR, n (%) | 30 (37.5%) |
| ORR (CR + PR), n (%) [95% CI] | 48 (60.0%) [48.4-70.8] |
| DCR (CR + PR + SD), n (%) [95% CI] | 59 (73.8%) [62.7-83.0] |

*NE patients had no valid post-baseline response assessments.
CI, confidence interval; CR, complete response; DCR, disease control rate; NE, not evaluable; ORR, objective response rate; PET, positron emission tomography; PD, progressive disease; PR, partial response; SD, stable disease.

The median duration of response was 21.7 months (95% CI, 21.7 months to not reached) and the 12-month duration of response rate was 71.6% (95% CI, 55.1 to 82.9%). Among patients achieving a complete response, the median duration of response has not yet been reached; the 12-month and 18-month duration of response rate was 93.2% (95% CI, 75.4 to 98.3%). Among patients achieving a partial response, median duration of response was 4.4 months (95% CI, 2.0 to 9.1 months).

Median progression-free survival was 12.1 months (95% CI, 5.7 months to not reached). Patients free from progression at 12 months (50.2% [95% CI, 37.9 to 61.2%]) tended to remain progression-free at 18 months (45.8% [95% CI, 33.4 to 57.4%]). Median progression-free survival after discontinuation of lenalidomide was 12.7 months (95% CI, 2.3 months to not reached). Median overall survival was not yet reached; 73.7% (95% CI, 62.2 to 82.2%) of patients were alive at 12 months.

### Safety

The median duration of exposure to study treatment was 9.3 months (range, 0.2 to 32.1 months); median duration of exposure to combination treatment or lenalidomide was 6.2 months (range, 0.1 to 12.5 months) and to MOR00208 monotherapy (following discontinuation of lenalidomide) was 4.1 months (range, 0.1 to 20.8 months).

Treatment-emergent adverse events occurred in 81 (100%) patients. The most common treatment-emergent adverse event (all grades) and the most common grade 3 or higher adverse event was neutropenia, occurring in 40 (49.4%) and 39 (48.1%) patients, respectively. Neutropenia was managed by granulocyte colony stimulating factor in 36 (44.4%) patients, and the majority (81% with grade ¾ neutropenia) recovered to baseline levels within 1 week. The next most common grade 3 or higher events were thrombocytopenia (14 [17.3%] patients), febrile neutropenia (10 [12.3%]), leukopenia (seven [8.6%]), anemia (six [7.4%]), and pneumonia/lung infection (six [7.4%]). The majority of non-hematologic adverse events were grade 1 and 2; diarrhea was the most common, occurring in 27 (33.3%) patients (nine [11.1%] with grade 2 and one [1.2%] with grade 3), and with a median duration of 8 days. Twenty-nine (35.8%) patients experienced different types of rash, most of which were grade 2 or lower. Infusion-related reactions (all grade 1) were observed in five (6.2%) patients. All occurred once during the first infusion and no interruption of infusion was required.

Serious adverse events occurred in 41/81 (50.6%) patients, of which 15/81 (18.5%) were suspected to be treatment-related by the investigators; these were primarily infections (eight [9.9%]) or febrile neutropenia (four [4.9%]).

In total, 14/81 (17.3%) patients discontinued lenalidomide and/or MOR00208 due to adverse events at any time during the study. Seven (8.6%) patients experienced an adverse event of special interest (defined by the protocol): three with tumor flares (one each at grade 1 to 3), one with grade 2 basal cell carcinoma, and three with grade 3 allergic dermatitis.

Thirty (37.0%) deaths were recorded, eight occurred during study treatment and 22 post-treatment. Twenty-three deaths were related to lymphoma progression and seven were unrelated to disease progression. Treatment-emergent adverse events leading to death occurred in four patients (sudden death, respiratory failure, cerebrovascular accident, and progressive multifocal leukoencephalopathy), none of which the investigators considered to be related to study treatment.

Upon discontinuation of lenalidomide (either cycle 13 onwards as per protocol or earlier in case of toxicities), the incidence and severity of treatment-emergent adverse events decreased under MOR00208 monotherapy; grade 3 or 4 neutropenia occurred in 6/51 (11.8%) patients during this phase. In total, adverse events of grade 3 or 4 were reported in 56/81 (70.0%) patients before lenalidomide discontinuation, compared with 15/51 (29.4%) patients after lenalidomide discontinuation.

### Conclusion

In this population of patients with relapsed or refractory DLBCL ineligible for stem cell transplant, combination treatment with MOR00208 and lenalidomide elicited an overall objective response in 60% of patients and a complete response in 42.5%. Furthermore, the responses were long-lasting, with a median duration of response of 21.7 months. Among patients with a complete response, the 18-month duration of response rate was 93.2%. With a median follow-up of nearly 20 months, median overall survival had not been reached. In the context of other recently reported drug trials in similar populations, our results indicate a promising treatment option; in particular as prior studies have reported objective response rates of 26% (SCHOLAR-1) (Blood 130, 1800-1808, 2017) 33% for lenalidomide plus rituximab, (Leukemia 27, 1902-1909, 2013), 25% for ibrutinib monotherapy (Nat. Med. 21, 922-926, 2015) and 28% for lenalidomide monotherapy (Clin. Cancer Res. 23, 4127-4137, 2017).

The L-MIND study indicates the benefit provided by the addition of MOR00208 to lenalidomide, given that single-agent lenalidomide has demonstrated objective response rates ranging from 27.5 to 35% in patients with relapsed or refractory aggressive non-Hodgkin's lymphoma (including DLBCL) (Clin. Cancer Res. 23, 4127-4137, 2017; Ann. Oncol. 22, 1622-1627, 2011; J. Clin. Oncol. 26, 4952-7, 2008) and single-agent MOR00208 has demonstrated an objective response rate of 26% in relapsed or refractory DLBCL (Ann. Oncol. 29, 1266-1272, 2018). The greater activity in L-MIND is most likely based on the complementary mechanism of action of both agents; the observed increase in NK cell numbers following treatment—as a result of a lenalidomide-mediated decreased activation threshold (Blood 126, 50-60, 2015)—may be a factor behind this synergy. CD19 appears to be a useful alternative target in patients who were not cured with prior anti-CD20-based immunochemotherapy, and a randomized phase 2/3 study is ongoing to explore the combination of MOR00208 with chemotherapy in patients previously exposed to rituximab (NCT02763319).

These data from this trial support the potential use of MOR00208 plus lenalidomide as an effective, well-tolerated chemotherapy-free option for treatment of patients with relapsed or refractory DLBCL ineligible for ASCT.

### Example 2: MOR00208 Plus Lenalidomide in Subgroups having Relapsed or Refractory Diffuse Large B-Cell Lymphoma

Of 81 patients enrolled, 80 patients received MOR00208+LEN and were included in the full analysis set (FAS) for efficacy. Median follow-up was 17.3 months. In the FAS, ORR was 60.0% (95% confidence interval [CI]: 48.4-70.8) (Table 3). The CR rate was 42.5% (n=34/80), of which 88.2% (n=30/34) were PET-confirmed. Median time to response (PR or CR) was 2.0 months and median time to CR was 7.1 months. Median DOR was 21.7 months (95% CI: 21.7- not reached [NR]); median PFS was 12.1 months (95% CI: 5.7-NR); and median OS was NR (95% CI: 18.3-NR) with a median follow-up of 19.6 months. The 12-month DOR and OS rates were 71.6% (95% CI: 55.1-82.9) (Table 3) and 73.7% (95% CI: 62.2-82.2) (Table 3), respectively.

In the subgroup analysis, patients with CR as best objective response (BOR) had better outcomes than those with PR: median DOR, NR (95% CI: 21.7-NR) vs 4.4 months (95% CI: 2.0-9.1); 12-month DOR rate, 93.2% (95% CI: 75.4-98.3) vs 14.4% (95% CI: 1.1-43.7); and 12-month OS rate, 97.1% vs 76.9%.

Patients with one prior line of therapy had a trend for better outcomes than those with ≥2 prior lines: ORR, 70.0% vs 50.0%; and 12-month OS rate, 86.9% vs 60.1%. However, the 12-month DOR rate was similar regardless of the number of prior lines (one prior line: 70.5% [95% CI: 47.2-85.0] vs ≥2 prior lines: 72.7% [95% CI: 46.3-87.6]).

For patients who were refractory to primary therapy or their last line of therapy, similar ORRs were observed to non-refractory patients (60.0% vs 60.0%); 12-month DOR was similar regardless of refractory status to last therapy; and 12-month OS rates were higher in non-refractory patients (Table 3).

As expected, patients with a low/low-intermediate International Prognostic Index (IPI) score had better outcomes than those with an intermediate-high/high score: ORR, 70.0% vs 50.0%; 12-month DOR rate, 86.5% vs 50.4%; and 12-month OS rate, 87.0% vs 59.9%.

Based on Hans algorithm, encouraging outcomes were reported in patients with germinal center B-cell (GCB) DLBCL (n=37), and outcomes were even better in those with non-GCB DLBCL (n=21): ORR, 48.6% vs 71.4%; 12-month DOR rate, 53.5% vs 83.1%; and 12-month OS rate, 65.4% vs 84.2% (Table 3). Given that single-agent lenalidomide is historically less active in the GCB subgroup, (Clin. Cancer Res. 23, 4127-4137, 2017; Oncologist 21, 1107-12, 2016; Annals of Oncology 26, 2015) these results suggest the greater activity and synergy of the MOR00208 plus lenalidomide combination.

TABLE 3
Subgroup analyses from L-MIND.

| | | ORR, | 12-month DOR | | 12-month PFS | | 12-month OS | |
|---|---|---|---|---|---|---|---|---|
| Subgroup | N | % (95% CI) | rate, % (95% CI) | | rate, % (95% CI) | | rate, % (95% CI) | |
| Full Analysis Set | 80 | 60.0 (48.4-70.8) | 71.6 | (55.1-82.9) | 50.2 | (37.9-61.2) | 73.7 | (62.2-82.2) |
| IPI score | | | | | | | | |
| Low/low-intermediate | 40 | 70.0 (53.5-83.4) | 86.5 | (62.9-95.6) | 68.6 | (51.1-80.9) | 87.0 | (71.6-94.4) |
| Intermediate-high/high | 40 | 50.0 (33.8-66.2) | 50.4 | (26.0-70.5) | 30.2 | (15.7-46.1) | 59.9 | (42.4-73.6) |
| Primary refractory | | | | | | | | |
| Yes | 15 | 60.0 (32.3-83.7) | 44.4 | (13.6-71.9) | 29.6 | (9.3-53.6) | 53.3 | (26.3-74.4) |
| No | 65 | 60.0 (47.1-72.0) | 77.8 | (58.6-88.9) | 54.7 | (40.7-66.8) | 78.6 | (66.0-87.0) |
| Refractory to last line | | | | | | | | |
| Yes | 35 | 60.0 (42.1-76.1) | 70.6 | (45.8-85.6) | 44.8 | (27.9-60.4) | 59.0 | (40.7-73.3) |
| No | 45 | 60.0 (44.3-74.3) | 71.7 | (46.8-86.5) | 54.5 | (36.9-69.1) | 85.6 | (70.6-93.2) |
| Number of prior lines | | | | | | | | |
| 1 | 41 | 70.7 (54.5-83.9) | 67.7 | (45.0-82.7) | 58.8 | (41.0-72.9) | 87.2 | (72.0-94.5) |
| ≥2 | 39 | 48.7 (32.4-65.2) | 77.0 | (49.7-90.7) | 40.8 | (24.3-56.6) | 59.0 | (41.4-73.0) |
| Cell of origin (by IHC) | | | | | | | | |
| GCB | 36 | 50.0 (82.9-67.1) | 53.5 | (27.9-73.6) | 37.0 | (20.5-53.5) | 64.4 | (45.8-78.0) |
| Non-GCB | 20 | 75.0 (50.9-91.3) | 83.1 | (47.2-95.5) | 73.0 | (46.7-87.8) | 83.3 | (56.8-94.3) |
| Best objective response | | | | | | | | |
| PR | 14 | N/A | 14.4 | (1.1-43.7) | 20.5 | (6.5-64.3) | 76.9 | (57.1-100) |
| CR | 34 | N/A | 93.2 | (75.4-98.3) | 97.0 | (91.3-100) | 97.1 | (91.5-100) |

DOR, duration of response; IHC, immunohistochemistry; GCB, germinal center B-cell; IPI, International Prognostic Index; N/A, not applicable; ORR, objective response rate; OS, overall survival; PFS, progression-free survival.

Overall, MOR00208+LEN combination followed by MOR00208 monotherapy shows encouraging activity with durable responses in ASCT-ineligible patients with R/R DLBCL. L-MIND includes a substantial number of poor prognosis patient subgroups. While the influence of these risk factors is evident, the clinical activity of MOR00208+LEN in these patients is promising, particularly in those who were refractory to prior therapies.

### Example 3: Updated MOR00208 Plus Lenalidomide Subgroups

Patients in the L-MIND study had a median age of 72 years (range 41-86) at enrollment and had received a median of 2 (range 1-4) prior lines of therapy. All patients had received R-CHOP or equivalent chemoimmunotherapy prior to study entry. Owing to the availability of additional data from a central pathology review of two patients, the baseline patient characteristics for cell of origin by immunohistochemistry and gene expression profiling have been updated since the primary analysis (Table 4). There was one patient of each with double- and triple-hit DLBCL.

Patient subgroups of clinical interest included 15 patients (18.5%) with primary refractory disease, 34 patients (42.0%) with rituximab refractory disease, and 36 patients (44.4%) who were refractory to their last therapy. Most patients who were refractory to their last line of therapy had received two prior lines of treatment (71.4%), and last prior line included chemotherapy in 94.4% and rituximab in 80.0% of patients. Baseline characteristics in refractory subgroups were generally comparable with the overall population (Table 4), although patients in refractory subgroups were more likely to have increased lactate dehydrogenase and germinal center B cell of origin by immunohistochemistry.

TABLE 4
Updated baseline characteristics and patient subgroups of clinical interest.

| | | | Primary | | | | | |
|---|---|---|---|---|---|---|---|---|
| | | | refractory | | Rituximab-refractory | | Last-therapy- | |
| | All patients | | disease | | disease | | refractory | |
| n | 81 | | 15 | | 34 | | 36 | |
| Median age, y (range) | 72 | (41-86) | 73 | (48-82) | 72.5 | (41-82) | 72.5 | (41-82) |
| Age >70 years, n (%) | 45 | (56) | 9 | (60) | 19 | (56) | 20 | (56) |
| Median prior lines | 2 | (1-4) | 2 | (1-4) | 2 | (1-4) | 2 | (1-4) |
| of treatment (range) | | | | | | | | |
| Stage III/IV, n (%) | 61 | (75) | 10 | (67) | 24 | (71) | 27 | (75) |
| increased LDH, n (%) | 45 | (56) | 10 | (67) | 22 | (65) | 25 | (69) |
| IPI 3-5, n (%) | 41 | (51) | 8 | (53) | 19 | (56) | 21 | (58) |
| Prior ASCT, n (%) | 9 | (11) | 0 | | 3 | (9) | 4 | (11) |
| Cell of origin (by IHC), n (%) | | | | | | | | |
| GCB | 39 | (48) | 12 | (80) | 21 | (62) | 21 | (58) |
| Non-GCB | 22 | (27) | 1 | (7) | 6 | (18) | 8 | (22) |
| Unknown | 20 | (25) | 2 | (13) | 7 | (21) | 7 | (19) |
| Cell of origin (gene expression | | | | | | | | |
| profiling), n (%) | | | | | | | | |
| GCB | 8 | (10) | 2 | (13) | 5 | (15) | 5 | (14) |
| ABC | 20 | (25) | 5 | (33) | 8 | (24) | 8 | (22) |
| Unclassified | 6 | (7) | 1 | (7) | 1 | (3) | 4 | (11) |
| Not evaluable | 5 | (6) | 2 | (13) | 3 | (9) | 3 | (8) |
| Missing | 42 | (52) | 5 | (33) | 17 | (50) | 16 | (44) |
| Patients with transformed | | | | | | | | |
| lymphoma,* n (%) | | | | | | | | |
| B-cell lymphoma (Follicular | 4 | (5) | 1 | (7) | 2 | (6) | 2 | (3) |
| Lymphoma) | | | | | | | | |

| | | | | | | | |
|---|---|---|---|---|---|---|---|
| Marginal zone lymphoma | 2 | (3) | 1 | (7) | 0 | 1 | (3) |

| NHL unspecified histology | 1 | (1) | 0 | 0 | 0 | |
|---|---|---|---|---|---|---|
| Case reported by central | 1 | (1) | 0 | 0 | 1 | (3) |
| pathology review | | | | | | |

From records in the medical history for seven patients with transformed lymphoma and as a current medical condition (ongoing at cycle 1, day 1) for one B-cell lymphoma patient. Refractory subgroups may overlap. Primary refractory disease defined as progression during first-line treatment and/or response of PD or SD to first-line treatment or PD within 6 months after completion of first-line treatment. Rituximab-refractory defined as PD or SD to any rituximab-containing regimen or PD during or within 6 months of completion of any rituximab-containing therapy line. Last therapy-refractory defined as PD or SD to most recently administered therapy before study entry.

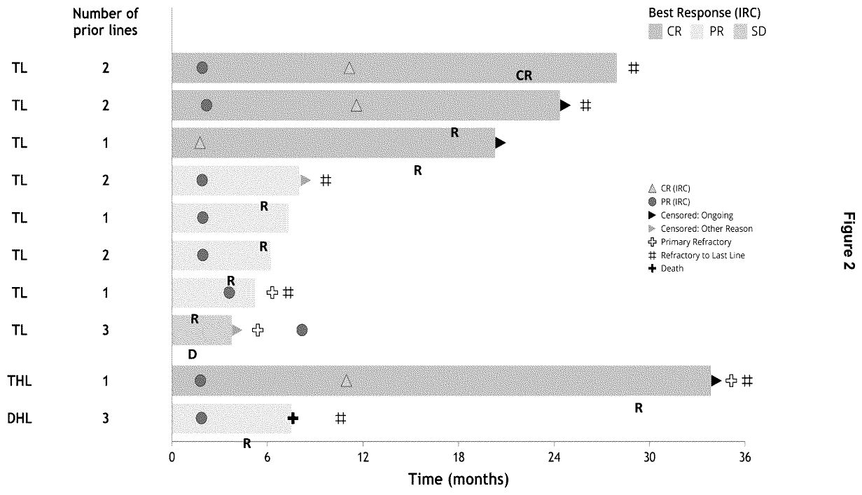

Based on medical history and central pathology diagnosis, eight patients had DLBCL arising from transformation of low-grade lymphoma, and there was one patient each with double-and triple-hit lymphoma. Of the eight patients with transformed lymphoma, four experienced PR and three experienced CR. The patient with double-hit lymphoma (MYC and BCL2 translocations) was refractory to his last line of therapy before L-MIND (R-dexamethasone-cytarabine-cisplatin) and achieved a PR. The patient with triple-hit lymphoma (MYC, BCL2 and BCL6 translocations) had previously experienced a CR for 4.5 months in response to R-CHOP and started tafasitamab plus lenalidomide 1 month after relapse. This patient experienced a CR in L-MIND with sustained remission for >30 months. Swimmer plots for all of these patients are shown in FIG. 2. Overall, two patients with double- and triple-hit lymphoma and seven out of eight patients with transformed lymphoma responded to therapy.

1. A method of treating a hematological cancer in human subjects in need thereof, comprising administering to the human subjects a therapeutic combination of an anti-CD19 antibody and lenalidomide wherein said treatment extends the overall survival and/or the progression free survival of said human subjects.

2.-3. (canceled)

4. The method of claim 1, wherein said human subjects have received one line of previous treatment and wherein the 12-months overall survival rate is 80% or more.

5. The method of claim 4, wherein the 12-months progression free survival is 55% or more.

6. The method of claim 1, wherein said human subjects have received two or more lines of previous treatment and wherein the 12-months overall survival rate is 55% or more.

7. The method of claim 6, wherein the 12-months progression free survival is 35% or more.

8. The method of claim 1, wherein said hematological cancer is a non-Hodgkin's lymphoma selected from the group consisting of follicular lymphoma, small lymphocytic lymphoma, mucosa-associated lymphoid tissue, marginal zone lymphoma, diffuse large B cell lymphoma (DLBCL), Burkitt's lymphoma and mantle cell lymphoma.

9. The method of claim 1, wherein said hematological cancer is a germinal center B-cell type (GCB) DLBCL and wherein the 12-months overall survival rate is 60% or more.

10. The method of claim 9, wherein the 12-months progression free survival is 35% or more.

11. The method of claim 1, wherein said hematological cancer is a non-germinal center B-cell type (non-GCB) DLBCL and wherein the 12-months overall survival rate is 80% or more.

12. The method of claim 11, wherein the 12-months progression free survival is 70% or more.

13. The method of claim 1, wherein said anti-CD19 antibody comprises an HCDR1 region comprising the sequence SYVMH (SEQ ID NO: 1), an HCDR2 region comprising the sequence NPYNDG (SEQ ID NO: 2), an HCDR3 region comprising the sequence GTYYYGTRVFDY (SEQ ID NO: 3), an LCDR1 region comprising the sequence RSSKSLQNVNGNTYLY (SEQ ID NO: 4), an LCDR2 region comprising the sequence RMSNLNS (SEQ ID NO: 5), and an LCDR3 region comprising the sequence MQHLEYPIT (SEQ ID NO: 6).

14. The method of claim 1, wherein said anti-CD19 antibody comprises a variable heavy chain of the sequence
(SEQ ID NO: 7)
EVQLVESGGGLVKPGGSLKLSCAASGYTFTSYVMHWVRQAPGKGLEWIGY
INPYNDGTKYNEKFQGRVTISSDKSISTAYMELSSLRSEDTAMYYCARGT
YYYGTRVFDYWGQGTLVTVSS
and a variable light chain of the sequence
(SEQ ID NO: 8)
DIVMTQSPATLSLSPGERATLSCRSSKSLQNVNGNTYLYWFQQKPGQSPQ
LLIYRMSNLNSGVPDRFSGSGSGTEFTLTISSLEPEDFAVYYCMQHLEYP
ITFGAGTKLEIK.

15. The method of claim 1, wherein said anti-CD19 antibody comprises a heavy chain having the sequence
(SEQ ID NO: 11)
EVQLVESGGGLVKPGGSLKLSCAASGYTFTSYVMHWVRQAPGKGLEWIGY
INPYNDGTKYNEKFQGRVTISSDKSISTAYMELSSLRSEDTAMYYCARGT
YYYGTRVFDYWGQGTLVTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKKVEPKSCDKTHTCPPCPAPELLGGPDVFLFPPK
PKDTLMISRTPEVTCVVVDVSHEDPEVQFNWYVDGVEVHNAKTKPREEQF
NSTFRVVSVLTVVHQDWLNGKEYKCKVSNKALPAPEEKTISKTKGQPREP
QVYTLPPSREEMTKNQVSLTCLVKGFYPSDIAVEWESNGQPENNYKTTPP
MLDSDGSFFLYSKLTVDKSRWQQGNVFSCSVMHEALHNHYTQKSLSLSPG
K
and a light chain having the sequence
(SEQ ID NO: 12)
DIVMTQSPATLSLSPGERATLSCRSSKSLQNVNGNTYLYWFQQKPGQSPQ
LLIYRMSNLNSGVPDRFSGSGSGTEFTLTISSLEPEDFAVYYCMQHLEYP
ITFGAGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC.

16. The method of claim 1, wherein said anti-CD19 antibody is administered at least bi-weekly at an amount of 12 mg/kg per dose and wherein lenalidomide is administered daily at an amount of 25 mg.

17. A method of treating primary refractory DLBCL in a human subject in need thereof, comprising administering to the human subject a therapeutically effective amount of an anti-CD19 antibody and lenalidomide.

18. The method of claim 17, wherein the anti-CD19 antibody comprises an HCDR1 region comprising the sequence SYVMH (SEQ ID NO:1), an HCDR2 region comprising the sequence NPYNDG (SEQ ID NO:2), an HCDR3 region comprising the sequence GTYYYGTRVFDY (SEQ ID NO:3), an LCDR1 region comprising the sequence RSSKSLQNVNGNTYLY (SEQ ID NO:4), an LCDR2 region comprising the sequence RMSNLNS (SEQ ID NO:5), and an LCDR3 region comprising the sequence MQHLEYPIT (SEQ ID NO:6).

19. A method of treating non-GCB DLBCL in a human subject in need thereof, comprising administering to the human subject a therapeutically effective amount of an anti-CD19 antibody and lenalidomide.

20. The method of claim 19, wherein the anti-CD19 antibody comprises an HCDR1 region comprising the sequence SYVMH (SEQ ID NO:1), an HCDR2 region comprising the sequence NPYNDG (SEQ ID NO:2), an HCDR3 region comprising the sequence GTYYYGTRVFDY (SEQ ID NO:3), an LCDR1 region comprising the sequence RSSKSLQNVNGNTYLY (SEQ ID NO:4), an LCDR2 region comprising the sequence RMSNLNS (SEQ ID NO:5), and an LCDR3 region comprising the sequence MQHLEYPIT (SEQ ID NO:6).

21. The method of claim 19, wherein the non-GCB DLBCL is non-GCB relapsed or refractory DLBCL.

22. A method for improving the objective response rate (ORR) to treatment in human subjects having relapsed or refractory diffuse large B-cell lymphoma (rrDLBCL), comprising administering to the human subjects a therapeutic combination of an anti-CD19 antibody and lenalidomide, wherein the human subjects have an ORR of at least 50% after treatment with the therapeutic combination.

23. The method of claim 22, wherein the anti-CD19 antibody comprises an HCDR1 region comprising the sequence SYVMH (SEQ ID NO:1), an HCDR2 region comprising the sequence NPYNDG (SEQ ID NO:2), an HCDR3 region comprising the sequence GTYYYGTRVFDY (SEQ ID NO:3), an LCDR1 region comprising the sequence RSSKSLQNVNGNTYLY (SEQ ID NO:4), an LCDR2 region comprising the sequence RMSNLNS (SEQ ID NO:5), and an LCDR3 region comprising the sequence MQHLEYPIT (SEQ ID NO:6).
